(19) United States
(12) Patent Application Publication
(10) Pub. No.: US 2005/0171195 A1
(43) Pub. Date: Aug. 4, 2005

(54) P2X7 antagonists for treating neuropathic pain

(76) Inventors: William A. Carroll, Evanston, IL (US); Arturo Perez Medrano, Grayslake, IL (US); Michael F. Jarvis, Vernon Hills, IL (US); Ying Wang, Plainsboro, NJ (US); Sridhar Peddi, Grayslake, IL (US)

Correspondence Address: ROBERT DEBERARDINE;ABBOTT LABORATORIES, 100 ABBOTT PARK ROAD, DEPT. 377/AP6A, ABBOTT PARK, IL, 60064-6008, US

(21) Appl. No.: 10/980,674

(22) Filed: Nov. 3, 2004

Related U.S. Application Data
(60) Provisional application No. 60/517,685, filed on Nov. 6, 2003.

(51) Int. Cl. A61K 31/44; A61K 31/16

(52) U.S. Cl. 514/464; 514/609

(57) ABSTRACT

The present invention discloses a method for treating neuropathic pain using compounds of formula I 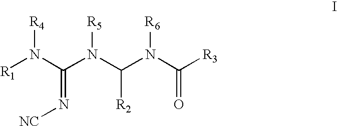 or compositions containing compounds of formula I.

The present application claims priority to U.S. Provisional Application Ser. No. 60/517,685, filed on Nov. 6, 2003, hereby incorporated in it entirety by reference.

### TECHNICAL FIELD

Antagonists of the P2X₇ receptor are useful in the treatment of neuropathic pain states.

### BACKGROUND OF THE INVENTION

P2X receptors are ionotropic receptors activated by ATP. The importance of P2X receptors in nociception is underscored by the variety of pain states in which this endogenous ligand can be released. Of the seven P2X receptors, the P2X₇ is distinguished by its ability to form a large pore upon prolonged or repeated agonist stimulation (Rassendren et al., J. Biol. Chem Vol. 272, pages 5482-5486, 1997). It is partially activated by saturating concentrations of ATP, whereas it is fully activated by the synthetic ATP analog benzoylbenzoic ATP (BzATP) (Bianchi et al., Eur. J. Pharmacol. Vol. 376, pages 127-138, 1999). The P2X₇ receptor is expressed by presynaptic terminals in the central and peripheral nervous systems, antigen-presenting cells including macrophages, human epidermal Langerhans' cells, microglial cells and a number of tumor cell lines of varying origin (Jacobson et al., “Adenosine and Adenine Nucleotides: From Molecular Biology to Integrative Physiology”. L. Belardinelli and A. Pelleg (eds.), Kluwer, Boston, pages 149-166, 1995).

On glial cells, the P2X₇ receptor has been shown to mediate release of glutamate (Anderson et al. Drug Dev. Res. Vol.50. page 92, 2000). Since glutamate is known to be involved in the neurotransmission of painful sensory signals, inhibition of P2X₇ may have therapeutic utility in the treatment of various pain states. Furthermore, oxidized ATP (oATP), a nonselective and irreversible P2X₇ antagonist, was recently reported to possess peripherally mediated antinociceptive properties in inflamed rats (Dell'Antonio et al., Neuroscience Lett., Vol. 327, pages 87-90, 2002). Thus, P2X₇ antagonists may have utility in the treatment of a variety of pain states.

Recent data also suggested a possible role for P2X₇ receptor activation in neuroinflammation and neurodegeneration. In the central nervous system, the P2X₇ receptor is predominately expressed by microglia, the resident macrophages of the brain. Upregulation of the P2X₇ receptor, most likely on activated microglia, was reported at the site of cerebral ischemic damage following middle cerebral artery occlusion in rat brain. Thus, P2X₇ antagonists may have utility in the treatment of neurodegenerative conditions including stroke and Alzheimer's disease (Collo et al., Neuropharmacology, Vol. 36, pages 1277-1283, 1997).

Activation of the P2X₇ receptor on cells of the immune system (macrophages, mast cells and lymphocytes) leads to release of interleukin-1β (IL-1β), giant cell formation, degranulation, and L-selectin shedding. Compounds acting at the P2X₇ receptor may therefore have utility in the treatment of various disease states and conditions such as rheumatoid arthritis, osteoarthritis, psoriasis, allergic dermatitis, asthma, chronic obstructive pulmonary disease, airways hyper-responsiveness, septic shock, glomerulonephritis, irritable bowel disease, Crohn's disease, ulcerative colitis, atherosclerosis, growth and metastases of malignant cells, myoblastic leukemia, diabetes, Alzheimer's disease, meningitis, osteoporosis, burn injury, ischemic heart disease, stroke and varicose veins.

Neuropathic pain is a type of pain different from pain involved with inflammatory or neurodegenerative conditions, it is associated with any disorder affecting any segment of the nervous system. Neuropathic pain is extremely difficult to manage; it is usually chronic and fails to respond to standard analgesic interventions. Administration of morphine may give some degree of relief but at doses that are impractical for lifelong treatments (Bennett, Hosp. Practice Vol. 33, pages 95 to 114, 1998). Common causes of neuropathic pain are, among others, alcoholism, amputation, cancer chemotherapy, diabetes, trigeminal neuralgia, HIV infection, multiple sclerosis, shingles and spine surgery. One of the most dramatic examples of neuropathic pain is called “phantom limb syndrome” which occurs when an arm or a leg have been removed, but the brain still gets pain messages from the missing limb. Since neuropathic pain is remarkably common, there is a need for P2X₇ antagonist that can be efficiently used in treating this neurological disorder.

A recent study has reported the localization of P2X₇ receptors on presynaptic terminals in the central and peripheral nervous systems. The activation of these receptors was linked to release of the neurotransmitter glutamate, which has a well established role in excitotoxicity and responses to injury (Deuchars et al J. Neuroscience, Vol. 21, pages 7143-7152, 2001). This finding indicates a role for the P2X₇receptor in the process of neuronal synaptic transmission and therefore a potential role for P2X₇ antagonists as novel therapeutic tool to treat neuropathic pain.

### SUMMARY OF THE INVENTION

In its principal embodiment, the present invention discloses a method for treating neuropathic pain in a mammal comprising administering to the mammal a compound having formula I, 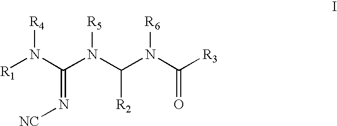
or a pharmaceutically acceptable salt, amide, ester or prodrug thereof, wherein
R₁ is selected from the group consisting of aryl, arylalkyl, heterocycle, and heterocyclealkyl;
R₂ is selected from the group consisting of alkyl and haloalkyl;
R₃ is selected from the group consisting of alkyl, aryl, arylalkyl, heterocycle and heterocyclealkyl; and
R₄, R₅, and R₆ are selected from the group consisting of hydrogen and alkyl.

Another embodiment of the present invention relates to a pharmaceutical composition comprising a compound of the present invention. Such compositions can be administered in accordance with a method of the invention, typically as part of a therapeutic regime for treatment or prevention of neuropathic pain. The compositions may contain one or more compounds of the present invention.

### DETAILED DESCRIPTION OF THE INVENTION

All patents, patent applications, and literature references cited in the specification are herein incorporated by reference in their entirety. In the case of inconsistencies, the present disclosure, including definitions, will prevail.

It is understood that the foregoing detailed description and accompanying examples are merely illustrative and are not to be taken as limitations upon the scope of the invention, which is defined solely by the appended claims and their equivalents. Various changes and modifications to the disclosed embodiments will be apparent to those skilled in the art. Such changes and modifications, including without limitation those relating to the chemical structures, substituents, derivatives, intermediates, syntheses, formulations and/or methods of use of the invention, may be made without departing from the spirit and scope thereof.

### DEFINITION OF TERMS

As used throughout this specification and the appended claims, the following terms have the following meanings.

The term “alkenyl” as used herein, means a straight or branched chain hydrocarbon containing from 2 to 10 carbons and containing at least one carbon-carbon double bond formed by the removal of two hydrogens. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, and 3-decenyl.

The term “alkoxy” as used herein, means an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy.

The term “alkoxycarbonyl” as used herein, means an alkoxy group, as defined herein, appended to the parent molecular moiety through a carbonyl group, as defined herein. Representative examples of alkoxycarbonyl include, but are not limited to, methoxycarbonyl, ethoxycarbonyl, and tert-butoxycarbonyl.

The term “alkoxycarbonylalkyl” as used herein, means an alkoxycarbonyl group, as defined herein, appended to the parent molecular moiety through an alkyl group, as defined herein. Representative examples of alkoxycarbonylalkyl include, but are not limited to, 3-methoxycarbonylpropyl, 4-ethoxycarbonylbutyl, and 2-tert-butoxycarbonylethyl.

The term “alkyl” as used herein, means a straight or branched chain hydrocarbon containing from 1 to 10 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

The term “alkylcarbonyl” as used herein, means an alkyl group, as defined herein, appended to the parent molecular moiety through a carbonyl group, as defined herein. Representative examples of alkylcarbonyl include, but are not limited to, acetyl, 1-oxopropyl, 2,2-dimethyl-1-oxopropyl, 1-oxobutyl, and 1-oxopentyl.

The term “alkylcarbonyloxy” as used herein, means an alkylcarbonyl group, as defined herein, appended to the parent molecular moiety through an oxygen atom. Representative examples of alkylcarbonyloxy include, but are not limited to, acetyloxy, ethylcarbonyloxy, and tert-butylcarbonyloxy.

The term “alkylcarbonyloxyalkyl” as used herein, means an alkylcarbonyloxy group, as defined herein, appended to the parent molecular moiety through an alkyl group, as defined herein. Representative examples of alkylcarbonyloxyalkyl include, but are not limited to, acetyloxymethyl, ethylcarbonyloxymethyl, and tert-butylcarbonyloxymethyl.

The term “alkylsulfinyl” as used herein, means an alkyl group, as defined herein, appended to the parent molecular moiety through a sulfinyl group, as defined herein. Representative examples of alkylsulfinyl include, but are not limited to, methylsulfinyl and ethylsulfinyl.

The term “alkylsulfonyl” as used herein, means an alkyl group, as defined herein, appended to the parent molecular moiety through a sulfonyl group, as defined herein. Representative examples of alkylsulfonyl include, but are not limited to, methylsulfonyl and ethylsulfonyl.

The term “alkylthio” as used herein, means an alkyl group, as defined herein, appended to the parent molecular moiety through a sulfur atom. Representative examples of alkylthio include, but are not limited, methylthio, ethylthio, tert-butylthio, and hexylthio.

The term “alkynyl” as used herein, means a straight or branched chain hydrocarbon group containing from 2 to 10 carbon atoms and containing at least one carbon-carbon triple bond. Representative examples of alkynyl include, but are not limited, to acetylenyl, 1-propynyl, 2-propynyl, 3-butynyl, 2-pentynyl, and 1-butynyl.

The term “aryl,” as used herein, refers to a monocyclic carbocyclic ring system or a bicyclic carbocyclic fused ring system having one or more aromatic rings. Representative examples of aryl include, azulenyl, indanyl, indenyl, naphthyl, phenyl, tetrahydronaphthyl, and the like. The aryl groups of this invention, including the representative examples listed above, can be optionally substituted with 1, 2, 3, 4, or 5 substituents independently selected from alkenyl, alkoxy, alkoxycarbonyl, alkoxycarbonylalkyl, alkyl, alkylcarbonyl, alkylcarbonyloxy, alkylcarbonyloxyalkyl, alkylsulfinyl, alkylsulfonyl, alkylthio, alkynyl, arylalkoxycarbonyl, arylalkoxycarbonylalkyl, arylcarbonyloxy, arylcarbonyloxyalkyl, aryloxycarbonyl, aryloxycarbonylalkyl, arylsulfonyl, carboxy, cyano, ethylenedioxy, formyl, halogen, haloalkyl, haloalkoxy, hydroxy, hydroxyalkyl, mercapto, mercaptoalkyl, methylenedioxy, nitro, R_{A}R_{B}N—, (R_{A}R_{B}N)alkyl, (R_{A}R_{B}N)carbonyl, (R_{A}R_{B}N)carbonylalkyl, (R_{A}R_{B}N)sulfonyl, (R_{A}R_{B}N)sulfonylalkyl, furyl, imidazolyl, isothiazolyl, isoxazolyl, naphthyl, oxadiazolyl, oxazolyl, phenyl, pyrazinyl, pyrazolyl, pyridinyl, pyrimidinyl, pyridazinyl, pyrrolyl, tetrazinyl, tetrazolyl, thiadiazolyl, thiazolyl, thienyl, triazinyl, triazolyl, benzimidazolyl, benzothiazolyl, benzothiadiazolyl, benzothienyl, benzoxadiazolyl, benzoxazolyl, benzofuranyl, cinnolinyl, indolyl, naphthyridinyl, isobenzofuranyl, isobenzothienyl, isoindolyl, isoquinolinyl, and quinolinyl, wherein said furyl, imidazolyl, isothiazolyl, isoxazolyl, naphthyl, oxadiazolyl, oxazolyl, phenyl, pyrazinyl, pyrazolyl, pyridinyl, pyrimidinyl, pyridazinyl, pyrrolyl, tetrazinyl, tetrazolyl, thiadiazolyl, thiazolyl, thienyl, triazinyl, triazolyl, benzimidazolyl, benzothiazolyl, benzothiadiazolyl, benzothienyl, benzoxadiazolyl, benzoxazolyl, benzofuranyl, cinnolinyl, indolyl, naphthyridinyl, isobenzofuranyl, isobenzothienyl, isoindolyl, isoquinolinyl, and quinolinyl may be substituted with 1 or 2 substituents independently selected from alkenyl, alkoxy, alkoxycarbonyl, alkoxycarbonylalkyl, alkyl, alkylcarbonyl, alkylcarbonyloxy, alkylcarbonyloxyalkyl, alkylsulfinyl, alkylsulfonyl, alkylthio, alkynyl, arylalkoxycarbonyl, arylalkoxycarbonylalkyl, arylcarbonyloxy, arylcarbonyloxyalkyl, aryloxycarbonyl, aryloxycarbonylalkyl, arylsulfonyl, carboxy, cyano, ethylenedioxy, formyl, halogen, haloalkyl, haloalkoxy, hydroxy, hydroxyalkyl, mercapto, mercaptoalkyl, methylenedioxy, nitro, R_{A}R_{B}N—, (R_{A}R_{B}N)alkyl, (R_{A}R_{B}N)carbonyl, (R_{A}R_{B}N)carbonylalkyl, (R_{A}R_{B}N)sulfonyl, and (R_{A}R_{B}N)sulfonylalkyl.

The term “arylalkoxy” as used herein, means an aryl group, as defined herein, appended to the parent molecular moiety through an alkoxy group, as defined herein. Representative examples of arylalkoxy include, but are not limited to, 2-phenylethoxy, 3-naphth-2-ylpropoxy, and 5-phenylpentyloxy.

The term “arylalkoxycarbonyl” as used herein, means an arylalkoxy group, as defined herein, appended to the parent molecular moiety through a carbonyl group, as defined herein. Representative examples of arylalkoxycarbonyl include, but are not limited to, benzyloxycarbonyl and naphth-2-ylmethoxycarbonyl.

The term “arylalkoxycarbonylalkyl” as used herein, means an arylalkoxycarbonyl group, as defined herein, appended to the parent molecular moiety through an alkyl group, as defined herein. Representative examples of arylalkoxycarbonylalkyl include, but are not limited to, benzyloxycarbonylmethyl and naphth-2-ylmethoxycarbonylmethyl.

The term “arylcarbonyl” as used herein, means an aryl group, as defined herein, appended to the parent molecular moiety through a carbonyl group, as defined herein. Representative examples of arylcarbonyl include, but are not limited to, benzoyl and naphthoyl.

The term “arylcarbonyloxy” as used herein, means an arylcarbonyl group, as defined herein, appended to the parent molecular moiety through an oxygen atom. Representative examples of arylcarbonyloxy include, but are not limited to, benzoyloxy and naphthoyloxy.

The term “arylcarbonyloxyalkyl” as used herein, means an arylcarbonyl group, as defined herein, appended to the parent molecular moiety through an oxygen atom. Representative examples of arylcarbonyloxy include, but are not limited to, benzoyloxymethyl and naphthoyloxymethyl.

The term “aryloxy” as used herein, means an aryl group, as defined herein, appended to the parent molecular moiety through an oxygen atom. Representative examples of aryloxy include, but are not limited to, phenoxy, naphthyloxy, 3-bromophenoxy, 4-chlorophenoxy, 4-methylphenoxy, and 3,5-dimethoxyphenoxy.

The term “aryloxycarbonyl” as used herein, means an aryloxy group, as defined herein, appended to the parent molecular moiety through a carbonyl group, as defined herein. Representative examples of aryloxycarbonyl include, but are not limited to, phenoxycarbonyl, naphthyloxycarbonyl, 3-bromophenoxycarbonyl, 4-chlorophenoxycarbonyl, 4-methylphenoxycarbonyl, and 3,5-dimethoxyphenoxycarbonyl.

The term “aryloxycarbonylalkyl” as used herein, means an aryloxycarbonyl group, as defined herein, appended to the parent molecular moiety through an alkyl group, as defined herein. Representative examples of aryloxycarbonylalkyl include, but are not limited to, phenoxycarbonylmethyl, naphthyloxycarbonylmethyl, 3-bromophenoxycarbonylmethyl, 4-chlorophenoxycarbonylmethyl, 4-methylphenoxycarbonylmethyl, and 3,5-dimethoxyphenoxycarbonylmethyl.

The term “arylsulfonyl” as used herein, means an aryl group, as defined herein, appended to the parent molecular moiety through a sulfonyl group, as defined herein. Representative examples of arylsulfonyl include, but are not limited to, phenylthio, naphthylthio, 3-bromophenylthio, 4-chlorophenylthio, 4-methylphenylthio, and 3,5-dimethoxyphenylthio.

The term “carbonyl” as used herein, means a —C(O)— group.

The term “carboxy” as used herein, means a —CO₂H group.

The term “cyano” as used herein, means a —CN group.

The term “ethylenedioxy” as used herein, means a —O(CH₂)₂O— group wherein the oxygen atoms of the ethylenedioxy group are attached to the two adjacent carbon atoms of an aryl group, as defined herein. A representative example includes, but is not limited to, dihydro-1,4-benzodixoinyl.

The term “formyl” as used herein, means a —C(O)H group.

The term “halo” or “halogen” as used herein, means —Cl, —Br, —I or —F.

The term “haloalkoxy” as used herein, means at least one halogen, as defined herein, appended to the parent molecular moiety through an alkoxy group, as defined herein. Representative examples of haloalkoxy include, but are not limited to, chloromethoxy, 2-fluoroethoxy, trifluoromethoxy, and pentafluoroethoxy.

The term “haloalkyl” as used herein, means at least one halogen, as defined herein, appended to the parent molecular moiety through an alkyl group, as defined herein. Representative examples of haloalkyl include, but are not limited to, chloromethyl, 2-fluoroethyl, trifluoromethyl, pentafluoroethyl, and 2-chloro-3-fluoropentyl.

The term “heterocycle,” as used herein, refers to a monocyclic or a bicyclic ring system. Monocyclic ring systems are exemplified by any 5 or 6 membered ring containing 1, 2, 3, or 4 heteroatoms independently selected from oxygen, nitrogen and sulfur. The 5-membered ring has from 0-2 double bonds and the 6-membered ring has from 0-3 double bonds. Representative examples of monocyclic ring systems include, but are not limited to, azetidinyl, azepanyl, aziridinyl, diazepanyl, 1,3-dioxolanyl, dioxanyl, 1,3-dioxanyl, dithianyl, furyl, imidazolyl, imidazolinyl, imidazolidinyl, isothiazolyl, isothiazolinyl, isothiazolidinyl, isoxazolyl, isoxazolinyl, isoxazolidinyl, morpholinyl, oxadiazolyl, oxadiazolinyl, oxadiazolidinyl, oxazolyl, oxazolinyl, oxazolidinyl, piperazinyl, piperidinyl, pyranyl, pyrazinyl, pyrazolyl, pyrazolinyl, pyrazolidinyl, pyridinyl, pyrimidinyl, pyridazinyl, pyrrolyl, pyrrolinyl, pyrrolidinyl, tetrahydrofuranyl, tetrahydrothienyl, tetrazinyl, tetrazolyl, thiadiazolyl, thiadiazolinyl, thiadiazolidinyl, thiazolyl, thiazolinyl, thiazolidinyl, thienyl, thiomorpholinyl, thiomorpholine sulfone, thiopyranyl, triazinyl, triazolyl, trithianyl, and the like. Bicyclic ring systems are exemplified by any of the above monocyclic ring systems fused to an aryl group as defined herein, a cycloalkyl group as defined herein, or another monocyclic ring system as defined herein. Representative examples of bicyclic ring systems include but are not limited to, for example, benzimidazolyl, benzothiazolyl, benzothiadiazolyl, benzothienyl, benzoxadiazolyl, benzoxazolyl, benzofuranyl, benzopyranyl, benzothiopyranyl, benzotriazolyl, benzodioxinyl, 1,3-benzodioxolyl, cinnolinyl, indazolyl, indolyl, indolinyl, indolizinyl, naphthyridinyl, isobenzofuranyl, isobenzothienyl, isoindolyl, isoindolinyl, 1-isoindolinonyl, isoquinolinyl, 1-isoquinolinonyl, phthalazinyl, pyranopyridinyl, quinolinyl, quinolizinyl, quinoxalinyl, quinazolinyl, tetrahydroisoquinolinyl, tetrahydroquinolinyl, and thiopyranopyridinyl.

The heterocycle groups of this invention, including the representative examples listed above, can be optionally substituted with 1, 2,or 3 substituents independently selected from alkenyl, alkoxy, alkoxycarbonyl, alkoxycarbonylalkyl, alkyl, alkylcarbonyl, alkylcarbonyloxy, alkylcarbonyloxyalkyl, alkylsulfinyl, alkylsulfonyl, alkylthio, alkynyl, arylalkoxycarbonyl, arylalkoxycarbonylalkyl, arylcarbonyloxy, arylcarbonyloxyalkyl, aryloxycarbonyl, aryloxycarbonylalkyl, arylsulfonyl, carboxy, cyano, formyl, halogen, haloalkyl, haloalkoxy, hydroxy, hydroxyalkyl, mercapto, mercaptoalkyl, nitro, oxo, R_{A}R_{B}N—, (R_{A}R_{B}N)alkyl, (R_{A}R_{B}N)carbonyl, (R_{A}R_{B}N)carbonylalkyl, (R_{A}R_{B}N)sulfonyl, (R_{A}R_{B}N)sulfonylalkyl, furyl, imidazolyl, isothiazolyl, isoxazolyl, naphthyl, oxadiazolyl, oxazolyl, phenyl, pyrazinyl, pyrazolyl, pyridinyl, pyrimidinyl, pyridazinyl, pyrrolyl, tetrazinyl, tetrazolyl, thiadiazolyl, thiazolyl, thienyl, triazinyl, triazolyl, benzimidazolyl, benzothiazolyl, benzothiadiazolyl, benzothienyl, benzoxadiazolyl, benzoxazolyl, benzofuranyl, cinnolinyl, indolyl, naphthyridinyl, isobenzofuranyl, isobenzothienyl, isoindolyl, isoquinolinyl, and quinolinyl wherein said furyl, imidazolyl, isothiazolyl, isoxazolyl, naphthyl, oxadiazolyl, oxazolyl, phenyl, pyrazinyl, pyrazolyl, pyridinyl, pyrimidinyl, pyridazinyl, pyrrolyl, tetrazinyl, tetrazolyl, thiadiazolyl, thiazolyl, thienyl, triazinyl, triazolyl, benzimidazolyl, benzothiazolyl, benzothiadiazolyl, benzothienyl, benzoxadiazolyl, benzoxazolyl, benzofuranyl, cinnolinyl, indolyl, naphthyridinyl, isobenzofuranyl, isobenzothienyl, isoindolyl, isoquinolinyl, and quinolinyl may be substituted with 1 or 2 substituents independently selected from alkenyl, alkoxy, alkoxycarbonyl, alkoxycarbonylalkyl, alkyl, alkylcarbonyl, alkylcarbonyloxy, alkylcarbonyloxyalkyl, alkylsulfinyl, alkylsulfonyl, alkylthio, alkynyl, arylalkoxycarbonyl, arylalkoxycarbonylalkyl, arylcarbonyloxy, arylcarbonyloxyalkyl, aryloxycarbonyl, aryloxycarbonylalkyl, arylsulfonyl, carboxy, cyano, formyl, halogen, haloalkyl, haloalkoxy, hydroxy, hydroxyalkyl, mercapto, mercaptoalkyl, nitro, R_{A}R_{B}N—, (R_{A}R_{B}N)alkyl, (R_{A}R_{B}N)carbonyl, (R_{A}R_{B}N)carbonylalkyl, (R_{A}R_{B}N)sulfonyl, and (R_{A}R_{B}N)sulfonylalkyl.

The term “hydroxy” as used herein, means an —OH group.

The term “hydroxyalkyl” as used herein, means at least one hydroxy group, as defined herein, is appended to the parent molecular moiety through an alkyl group, as defined herein. Representative examples of hydroxyalkyl include, but are not limited to, hydroxymethyl, 2-hydroxyethyl, 3-hydroxypropyl, 2,3-dihydroxypentyl, and 2-ethyl-4-hydroxyheptyl.

The term “mercapto” as used herein, means a —SH group.

The term “mercaptoalkyl” as used herein, means a mercapto group, as defined herein, appended to the parent molecular moiety through an alkyl group, as defined herein. Representative examples of mercaptoalkyl include, but are not limited to, 2-mercaptoethyl and 3-mercaptopropyl.

The term “nitro” as used herein, means a —NO₂ group.

The term “R_{A}R_{B}N—” as used herein, means two groups, R_{A} and R_{B}, which are appended to the parent molecular moiety through a nitrogen atom. R_{A} and R_{B} are each independently selected from the group consisting of hydrogen and alkyl. Representative examples of R_{A}R_{B}N— include, but are not limited to, amino, methylamino, acetylamino, and acetylmethylamino.

The term “(R_{A}R_{B}N)alkyl” as used herein, means a R_{A}R_{B}N— group, as defined herein, appended to the parent molecular moiety through an alkyl group, as defined herein. Representative examples of (R_{A}R_{B}N)alkyl include, but are not limited to, aminomethyl, 2-(methylamino)ethyl, 2-(dimethylamino)ethyl, and 3-(ethylmethylamino)propyl.

The term “(R_{A}R_{B}N)carbonyl” as used herein, means a R_{A}R_{B}N— group, as defined herein, appended to the parent molecular moiety through a carbonyl group, as defined herein. Representative examples of (R_{A}R_{B}N)carbonyl include, but are not limited to, aminocarbonyl, (methylamino)carbonyl, (dimethylamino)carbonyl, and (ethylmethylamino)carbonyl.

The term “(R_{A}R_{B}N)sulfonyl” as used herein, means a R_{A}R_{B}N— group, as defined herein, appended to the parent molecular moiety through a sulfonyl group, as defined herein. Representative examples of (R_{A}R_{B}N)sulfonyl include, but are not limited to, aminosulfonyl, (methylamino)sulfonyl, (dimethylamino)sulfonyl, and (ethylmethylamino)sulfonyl.

The term “(R_{A}R_{B}N)sulfonylalkyl” as used herein, means a (R_{A}R_{B}N)sulfonyl group, as defined herein, appended to the parent molecular moiety through an alkyl group, as defined herein. Representative examples of (R_{A}R_{B}N)sulfonylalkyl include, but are not limited to, aminosulfonylmethyl, (methylamino)sulfonylmethyl, (dimethylamino)sulfonylmethyl, and (ethylmethylamino)sulfonylmethyl.

The term “oxo” as used herein, means a ═O moiety.

The term “sulfinyl” as used herein, means a —S(O)— group.

The term “sulfonyl” as used herein, means a —SO₂— group.

Compounds of the present invention may exist as stereoisomers wherein, asymmetric or chiral centers are present. These stereoisomers are “R” or “S” depending on the configuration of substituents around the chiral carbon atom. The terms “R” and “S” used herein are configurations as defined in IUPAC 1974 Recommendations for Section E, Fundamental Stereochemistry, Pure Appl. Chem., 1976, 45: 13-30. The present invention contemplates various stereoisomers and mixtures thereof that are specifically included within the scope of this invention. Stereoisomers include enantiomers and diastereomers, and mixtures of enantiomers or diastereomers. Individual stereoisomers of compounds of the present invention may be prepared synthetically from commercially available starting materials that contain asymmetric or chiral centers or by preparation of racemic mixtures followed by resolution well known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary or (2) direct separation of the mixture of optical enantiomers on chiral chromatographic columns.

### EMBODIMENTS OF THE INVENTION

In its principal embodiment, the present invention discloses a method for treating neuropathic pain in a mammal comprising administering to the mammal a compound having formula I 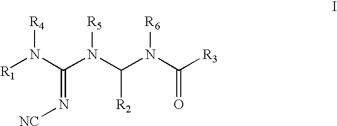
or a pharmaceutically acceptable salt, amide, ester or prodrug thereof, in which R₁ is selected from the group consisting of aryl, arylalkyl, heterocycle and heterocyclealkyl; R₂ is selected from the group consisting of alkyl, and haloalkyl; R₃ is selected from the group consisting of alkyl, aryl, arylalkyl, heterocycle and heterocyclealkyl; and R₄, R₅, and R₆ are selected from the group consisting of hydrogen and alkyl.

In another embodiment, the present invention discloses a method for treating neuropathic pain in a mammal comprising administering to the mammal a compound having formula I in which R₁ is aryl; R₂ is alkyl; R₃ is arylalkyl; and R₄, R₅, and R₆ are as defined in formula I.

In another embodiment, the present invention discloses a method for treating neuropathic pain in a mammal comprising administering to the mammal a compound having formula I wherein R₁ is aryl wherein the aryl is phenyl optionally substituted with 1, 2, or 3 substituents independently selected from the group consisting of alkyl and halogen; R₂ is alkyl wherein the alkyl is tert-butyl; R₃ is arylalkyl wherein the aryl of the arylalkyl is selected from the group consisting of naphthyl and phenyl, wherein the phenyl is optionally substituted with 1 or 2 substituents independently selected from the group consisting of alkoxy, alkyl, alkylthio, cyano, ethylenedioxy, halogen, methylenedioxy, and R_{A}R_{B}N—; and R_{A}, R_{B}, R₄, R₅, and R₆ are as defined in formula I.

In another embodiment, the present invention discloses a method for treating neuropathic pain in a mammal comprising administering to the mammal a compound having formula I wherein R₁ is aryl wherein the aryl is phenyl optionally substituted with 1, 2, or 3 substituents independently selected from the group consisting of alkyl and halogen; R₂ is alkyl wherein the alkyl is tert-butyl; R₃ is arylalkyl wherein the aryl of the arylalkyl is phenyl optionally substituted with 1 or 2 substituents independently selected from the group consisting of alkoxy, alkylthio, halogen, methylenedioxy; and R₄, R₅, and R₆ are hydrogen;

In another embodiment, the present invention discloses a method for treating neuropathic pain in a mammal comprising administering to the mammal a compound having formula I wherein R₁ is aryl; R₂ is haloalkyl; R₃ is arylalkyl; and R₄, R₅, and R₆ are as defined in formula I.

In another embodiment, the present invention discloses a method for treating neuropathic pain in a mammal comprising administering to the mammal a compound having formula I wherein R₁ is aryl wherein the aryl is phenyl optionally substituted with 1, 2, or 3 substituents independently selected from the group consisting of alkyl and halogen; R₂ is haloalkyl wherein the haloalkyl is 1,1-dichloroethyl; R₃ is arylalkyl wherein the aryl of the arylalkyl is selected from the group consisting of naphthyl and phenyl, wherein the phenyl is optionally substituted with 1 or 2 substituents independently selected from the group consisting of alkoxy, alkyl, alkylthio, cyano, ethylenedioxy, halogen, methylenedioxy, and R_{A}R_{B}N—; and R_{A}, R_{B}, R₄, R₅, and R₆ are as defined in formula I.

In another embodiment, the present invention discloses a method for treating neuropathic pain in a mammal comprising administering to the mammal a compound having formula I wherein R₁ is aryl; R₃ is alkyl; and R₂, R₄, R₅, and R₆ are as defined in formula I.

In another embodiment, the present invention discloses a method for treating neuropathic pain in a mammal comprising administering to the mammal a compound having formula I wherein R₁ is aryl wherein the aryl is phenyl optionally substituted with 1, 2, or 3 substituents independently selected from the group consisting of alkyl and halogen; R₂ is selected from the group consisting of tert-butyl and 1,1-dichloroethyl; R₃ is alkyl; and R₄, R₅, and R₆ are as defined in formula I.

In another embodiment, the present invention discloses a method for treating neuropathic pain in a mammal comprising administering to the mammal a compound having formula I wherein R₁ is aryl; R₃ is aryl; and R₂, R₄, R₅, and R₆ are as defined in formula I.

In another embodiment, the present invention discloses a method for treating neuropathic pain in a mammal comprising administering to the mammal a compound having formula I wherein R₁ is aryl wherein the aryl is phenyl optionally substituted with 1, 2, or 3 substituents independently selected from the group consisting of alkyl and halogen; R₂ is selected from the group consisting of tert-butyl and 1,1-dichloroethyl; R₃ is aryl wherein the aryl is selected from the group consisting of naphthyl and phenyl, wherein the phenyl is optionally substituted with 1 or 2 substituents independently selected from the group consisting of alkoxy, alkyl, alkylthio, cyano, ethylenedioxy, halogen, methylenedioxy, and R_{A}R_{B}N—; and R_{A}, R_{B}, R₄, R₅, and R₆ are as defined in formula I.

In another embodiment, the present invention discloses a method for treating neuropathic pain in a mammal comprising administering to the mammal a compound having formula I wherein R₁ is aryl; R₃ is heterocycle; and R₂, R₄, R₅, and R₆ are as defined in formula I.

In another embodiment, the present invention discloses a method for treating neuropathic pain in a mammal comprising administering to the mammal a compound having formula I wherein R₁ is aryl wherein the aryl is phenyl optionally substituted with 1, 2, or 3 substituents independently selected from the group consisting of alkyl and halogen; R₂ is selected from the group consisting of tert-butyl and 1,1-dichloroethyl; R₃ is heterocycle wherein the heterocycle is selected from the group consisting of pyridinyl, quinolinyl, and thienyl; and R₄, R₅, and R₆ are as defined in formula I.

In another embodiment, the present invention discloses a method for treating neuropathic pain in a mammal comprising administering to the mammal a compound having formula I wherein R₁ is aryl; R₃ is heterocyclealkyl; and R₂, R₄, R₅, and R₆ are as defined in formula I.

In another embodiment, the present invention discloses a method for treating neuropathic pain in a mammal comprising administering to the mammal a compound having formula I wherein R₁ is aryl wherein the aryl is phenyl optionally substituted with 1, 2, or 3 substituents independently selected from the group consisting of alkyl and halogen; R₂ is selected from the group consisting of tert-butyl and 1,1-dichloroethyl; R₃ is heterocyclealkyl wherein the heterocycle of the heterocyclealkyl is selected from the group consisting of pyridinyl, quinolinyl, and thienyl; and R₄, R₅, and R₆ are as defined in formula I.

In another embodiment, the present invention discloses a method for treating neuropathic pain in a mammal comprising administering to the mammal a compound having formula I wherein R₁ is heterocycle; R₂ is alkyl; R₃ is arylalkyl; and R₄, R₅, and R₆ are as defined in formula I.

In another embodiment, the present invention discloses a method for treating neuropathic pain in a mammal comprising administering to the mammal a compound having formula I wherein R₁ is heterocycle wherein the heterocycle is selected from the group consisting of benzoisothiazolyl, isoquinolinyl, pyridinyl, quinolinyl, and tetrahydroquinolinyl, wherein the heterocycle is optionally substituted with 1 or 2 substituents independently selected from the group consisting of alkyl, alkoxy, and halogen; R₂ is alkyl wherein the alkyl is tert-butyl; R₃ is arylalkyl wherein the aryl of the arylalkyl is selected from the group consisting of naphthyl and phenyl, wherein the phenyl is optionally substituted with 1 or 2 substituents independently selected from the group consisting of alkoxy, alkyl, alkylthio, cyano, ethylenedioxy, halogen, methylenedioxy, and R_{A}R_{B}N—; and R_{A}, R_{B}, R₄, R₅, and R₆ are as defined in formula I.

In another embodiment, the present invention discloses a method for treating neuropathic pain in a mammal comprising administering to the mammal a compound having formula I wherein R₁ is heterocycle wherein the heterocycle is selected from the group consisting of benzoisothiazolyl, isoquinolinyl, pyridinyl, quinolinyl, and tetrahydroquinolinyl, wherein the heterocycle is optionally substituted with 1 or 2 substituents independently selected from the group consisting of alkyl, alkoxy, and halogen; R₂ is alkyl wherein the alkyl is tert-butyl; R₃ is arylalkyl wherein the aryl of the arylalkyl is selected from the group consisting of naphthyl and phenyl, wherein the phenyl is optionally substituted with 1 or 2 substituents independently selected from the group consisting of alkoxy, alkyl, alkylthio, cyano, ethylenedioxy, halogen, and R_{A}R_{B}N—; R₄, R₅, and R₆ are hydrogen; and R_{A} and R_{B} are independently selected from the group consisting of hydrogen and alkyl.

In another embodiment, the present invention discloses a method for treating neuropathic pain in a mammal comprising administering to the mammal a compound having formula I wherein R₁ is heterocycle; R₂ is haloalkyl; R₃ is arylalkyl; and R₄, R₅, and R₆ are as defined in formula I.

In another embodiment, the present invention discloses a method for treating neuropathic pain in a mammal comprising administering to the mammal a compound having formula I wherein R₁ is heterocycle wherein the heterocycle is selected from the group consisting of benzoisothiazolyl, isoquinolinyl, pyridinyl, quinolinyl, and tetrahydroquinolinyl, wherein the heterocycle is optionally substituted with 1 or 2 substituents independently selected from the group consisting of alkoxy, alkyl, and halogen; R₂ is haloalkyl; R₃ is arylalkyl wherein the aryl of the arylalkyl is selected from the group consisting of naphthyl and phenyl, wherein the phenyl is optionally substituted with 1 or 2 substituents independently selected from the group consisting of alkoxy, alkyl, alkylthio, cyano, ethylenedioxy, halogen, methylenedioxy, and R_{A}R_{B}N—; and R_{A}, R_{B}, R₄, R₅, and R₆ are as defined in formula I.

In another embodiment, the present invention discloses a method for treating neuropathic pain in a mammal comprising administering to the mammal a compound having formula I wherein R₁ is heterocycle wherein the heterocycle is pyridinyl optionally substituted with 1 or 2 substituents independently selected from the group consisting of alkoxy and halogen; R₂ is haloalkyl wherein the haloalkyl is 1,1-dichloroethyl; R₃ is arylalkyl wherein the aryl of the arylalkyl is phenyl optionally substituted with 1 or 2 substituents independently selected from the group consisting of alkoxy, alkyl, alkylthio, cyano, ethylenedioxy, halogen, methylenedioxy, and R_{A}R_{B}N—; and R_{A}, R_{B}, R₄, R₅, and R₆ are as defined in formula I.

In another embodiment, the present invention discloses a method for treating neuropathic pain in a mammal comprising administering to the mammal a compound having formula I wherein R₁ is heterocycle wherein the heterocycle is pyridinyl optionally substituted with 1 alkoxy substituent; R₂ is haloalkyl wherein the haloalkyl is 1,1-dichloroethyl; R₃ is arylalkyl wherein the aryl of the arylalkyl is phenyl optionally substituted with 1 or 2 alkoxy substituents; and R₄, R₅, and R₆ are as defined in formula I.

In another embodiment, the present invention discloses a method for treating neuropathic pain in a mammal comprising administering to the mammal a compound having formula I wherein R₁ is heterocycle; R₂ is alkyl; R₃ is heterocyclealkyl; and R₄, R₅, and R₆ are as defined in formula I.

In another embodiment, the present invention discloses a method for treating neuropathic pain in a mammal comprising administering to the mammal a compound having formula I wherein R₁ is heterocycle wherein the heterocycle is selected from the group consisting of benzoisothiazolyl, isoquinolinyl, pyridinyl, quinolinyl, and tetrahydroquinolinyl, wherein the heterocycle is optionally substituted with 1 or 2 substituents independently selected from the group consisting of alkoxy, alkyl, and halogen; R₂ is alkyl; R₃ is heterocyclealkyl wherein the heterocycle of the heterocyclalkyl is selected from the group consisting of pyridinyl, quinolinyl, and thienyl; and R₄, R₅, and R₆ are as defined in formula I.

In another embodiment, the present invention discloses a method for treating neuropathic pain in a mammal comprising administering to the mammal a compound having formula I wherein R₁ is heterocycle wherein the heterocycle is selected from the group consisting of pyridinyl and quinolinyl, wherein the heterocycle is optionally substituted with 1 alkyl substituent; R₂ is alkyl wherein the alkyl is tert-butyl; R₃ is heterocyclealkyl wherein the heterocycle of the heterocyclalkyl is selected from the group consisting of pyridinyl, quinolinyl, and thienyl; and R₄, R₅, and R₆ are as defined in formula I.

In another embodiment, the present invention discloses a method for treating neuropathic pain in a mammal comprising administering to the mammal a compound having formula I wherein R₁ is heterocycle; R₂ is haloalkyl; R₃ is heterocyclealkyl; and R₄, R₅, and R₆ are as defined in formula I.

In another embodiment, the present invention discloses a method for treating neuropathic pain in a mammal comprising administering to the mammal a compound having formula I wherein R₁ is heterocycle wherein the heterocycle is selected from the group consisting of benzoisothiazolyl, isoquinolinyl, pyridinyl, quinolinyl, and tetrahydroquinolinyl, wherein the heterocycle is optionally substituted with 1 or 2 substituents independently selected from the group consisting of alkoxy, alkyl, and halogen; R₂ is haloalkyl wherein the haloalkyl is 1,1-dichloroethyl; R₃ is heterocyclealkyl wherein the heterocycle of the heterocyclalkyl is selected from the group consisting of pyridinyl, quinolinyl, and thienyl; and R₄, R₅, and R₆ are as defined in formula I.

In another embodiment, the present invention discloses a method for treating neuropathic pain in a mammal comprising administering to the mammal a compound having formula I wherein R₁ is heterocycle; R₂ is alkyl; R₃ is aryl; and R₄, R₅, and R₆ are as defined in formula I.

In another embodiment, the present invention discloses a method for treating neuropathic pain in a mammal comprising administering to the mammal a compound having formula I wherein R₁ is heterocycle wherein the heterocycle is selected from the group consisting of benzoisothiazolyl, isoquinolinyl, pyridinyl, quinolinyl, and tetrahydroquinolinyl, wherein the heterocycle is optionally substituted with 1 or 2 substituents independently selected from the group consisting of alkoxy, alkyl, and halogen; R₂ is alkyl wherein the alkyl is tert-butyl; R₃ is aryl wherein the aryl is selected from the group consisting of naphthyl and phenyl, wherein the phenyl is optionally substituted with 1 or 2 substituents independently selected from the group consisting of alkoxy, alkyl, alkylthio, cyano, ethylenedioxy, halogen, methylenedioxy, and R_{A}R_{B}N—; and R_{A}, R_{B}, R₄, R₅, and R₆ are as defined in formula I.

In another embodiment, the present invention discloses a method for treating neuropathic pain in a mammal comprising administering to the mammal a compound having formula I wherein R₁ is heterocycle; R₂ is haloalkyl; R₃ is aryl; and R₄, R₅, and R₆ are as defined in formula I.

In another embodiment, the present invention discloses a method for treating neuropathic pain in a mammal comprising administering to the mammal a compound having formula I wherein R₁ is heterocycle wherein the heterocycle is selected from the group consisting of benzoisothiazolyl, isoquinolinyl, pyridinyl, quinolinyl, and tetrahydroquinolinyl, wherein the heterocycle is optionally substituted with 1 or 2 substituents independently selected from the group consisting of alkoxy, alkyl, and halogen; R₂ is haloalkyl; R₃ is aryl wherein the aryl is selected from the group consisting of naphthyl and phenyl, wherein the phenyl is optionally substituted with 1 or 2 substituents independently selected from the group consisting of alkoxy, alkyl, alkylthio, cyano, ethylenedioxy, halogen, methylenedioxy, and R_{A}R_{B}N—; and R_{A}, R_{B}, R₄, R₅, and R₆ are as defined in formula I.

In another embodiment, the present invention discloses a method for treating neuropathic pain in a mammal comprising administering to the mammal a compound having formula I wherein R₁ is heterocycle wherein the heterocycle is selected from the group consisting of isoquinolinyl and pyridinyl, wherein the heterocycle is optionally substituted with 1 or 2 substituents independently selected from the group consisting of alkoxy and halogen; R₂ is haloalkyl wherein the haloalkyl is 1,1-dichloroethyl; R₃ is aryl wherein the aryl is selected from the group consisting of naphthyl and phenyl, wherein the phenyl is optionally substituted with 1 or 2 substituents independently selected from the group consisting of alkoxy, alkyl, alkylthio, cyano, ethylenedioxy, halogen, methylenedioxy, and R_{A}R_{B}N—; and R_{A}, R_{B}, R₄, R₅, and R₆ are as defined in formula I.

In another embodiment, the present invention discloses a method for treating neuropathic pain in a mammal comprising administering to the mammal a compound having formula I wherein R₁ is heterocycle wherein the heterocycle is selected from the group consisting of isoquinolinyl and pyridinyl, wherein the heterocycle is optionally substituted with 1 or 2 substituents independently selected from the group consisting of alkoxy and halogen; R₂ is haloalkyl wherein the haloalkyl is 1,1-dichloroethyl; R₃ is aryl wherein the aryl is phenyl optionally substituted with 1 or 2 substituents independently selected from the group consisting of alkoxy and halogen; and R₄, R₅, and R₆ are hydrogen.

In another embodiment, the present invention discloses a method for treating neuropathic pain in a mammal comprising administering to the mammal a compound having formula I wherein R₁ is heterocycle; R₃ is heterocycle; and R₂, R₄, R₅, and R₆ are as defined in formula I.

In another embodiment, the present invention discloses a method for treating neuropathic pain in a mammal comprising administering to the mammal a compound having formula I wherein R₁ is heterocycle wherein the heterocycle is selected from the group consisting of benzoisothiazolyl, isoquinolinyl, pyridinyl, quinolinyl, and tetrahydroquinolinyl, wherein the heterocycle is optionally substituted with 1 or 2 substituents independently selected from the group consisting of alkoxy, alkyl, and halogen; R₂ is selected from the group consisting of tert-butyl and 1,1-dichloroethyl; R₃ is heterocycle wherein the heterocycle is selected from the group consisting of pyridinyl, quinolinyl, and thienyl; and R₄, R₅, and R₆ are as defined in formula I.

In another embodiment, the present invention discloses a method for treating neuropathic pain in a mammal comprising administering to the mammal a compound having formula I wherein R₁ is heterocycle; R₃ is alkyl; and R₂, R₄, R₅, and R₆ are as defined in formula I.

In another embodiment, the present invention discloses a method for treating neuropathic pain in a mammal comprising administering to the mammal a compound having formula I wherein R₁ is heterocycle wherein the heterocycle is selected from the group consisting of benzoisothiazolyl, isoquinolinyl, pyridinyl, quinolinyl, and tetrahydroquinolinyl, wherein the heterocycle is optionally substituted with 1 or 2 substituents independently selected from the group consisting of alkoxy, alkyl, and halogen; R₂ is selected from the group consisting of tert-butyl and 1,1-dichloroethyl; R₃ is alkyl; and R₄, R₅, and R₆ are as defined in formula I.

In another embodiment, the present invention discloses a method for treating neuropathic pain in a mammal comprising administering to the mammal a compound having formula I wherein R₁ is heterocyclealkyl; R₃ is alkyl; and R₂, R₄, R₅, and R₆ are as defined in formula I.

In another embodiment, the present invention discloses a method for treating neuropathic pain in a mammal comprising administering to the mammal a compound having formula I wherein R₁ is heterocyclealkyl wherein the heterocycle of the heterocyclealkyl is selected from the group consisting of benzoisothiazolyl, isoquinolinyl, pyridinyl, quinolinyl, and tetrahydroquinolinyl, wherein the heterocycle is optionally substituted with 1 or 2 substituents independently selected from the group consisting of alkoxy, alkyl, and halogen; R₂ is selected from the group consisting of tert-butyl and 1,1-dichloroethyl; R₃ is alkyl; and R₄, R₅, and R₆ are as defined in formula I.

In another embodiment, the present invention discloses a method for treating neuropathic pain in a mammal comprising administering to the mammal a compound having formula I wherein R₁ is heterocyclealkyl; R₃ is aryl; and R₂, R₄, R₅, and R₆ are as defined in formula I.

In another embodiment, the present invention discloses a method for treating neuropathic pain in a mammal comprising administering to the mammal a compound having formula I wherein R₁ is heterocyclealkyl wherein the heterocycle of the heterocyclealkyl is selected from the group consisting of benzoisothiazolyl, isoquinolinyl, pyridinyl, quinolinyl, and tetrahydroquinolinyl, wherein the heterocycle is optionally substituted with 1 or 2 substituents independently selected from the group consisting of alkoxy, alkyl, and halogen; R₂ is selected from the group consisting of tert-butyl and 1,1-dichloroethyl; R₃ is aryl wherein the aryl is selected from the group consisting of naphthyl and phenyl, wherein the phenyl is optionally substituted with 1, 2, or 3 substituents independently selected from the group consisting of alkoxy, alkyl, alkylthio, cyano, ethylenedioxy, halogen, methylenedioxy, and R_{A}R_{B}N—; and R_{A}, R_{B}, R₄, R₅, and R₆ are as defined in formula I.

In another embodiment, the present invention discloses a method for treating neuropathic pain in a mammal comprising administering to the mammal a compound having formula I wherein R₁ is heterocyclealkyl; R₃ is arylalkyl; and R₂, R₄, R₅, and R₆ are as defined in formula I.

In another embodiment, the present invention discloses a method for treating neuropathic pain in a mammal comprising administering to the mammal a compound having formula I wherein R₁ is heterocyclealkyl wherein the heterocycle of the heterocyclealkyl is selected from the group consisting of benzoisothiazolyl, isoquinolinyl, pyridinyl, quinolinyl, and tetrahydroquinolinyl, wherein the heterocycle is optionally substituted with 1 or 2 substituents independently selected from the group consisting of alkoxy, alkyl, and halogen; R₂ is selected from the group consisting of tert-butyl and 1,1-dichloroethyl; R₃ is arylalkyl wherein the aryl of the arylalkyl is selected from the group consisting of naphthyl and phenyl, wherein the phenyl is optionally substituted with 1, 2, or 3 substituents independently selected from the group consisting of alkoxy, alkyl, alkylthio, cyano, ethylenedioxy, halogen, methylenedioxy, and R_{A}R_{B}N—; and R_{A}, R_{B}, R₄, R₅, and R₆ are as defined in formula I.

In another embodiment, the present invention discloses a method for treating neuropathic pain in a mammal comprising administering to the mammal a compound having formula I wherein R₁ is heterocyclealkyl; R₃ is heterocyclealkyl; and R₂, R₄, R₅, and R₆ are as defined in formula I.

In another embodiment, the present invention discloses a method for treating neuropathic pain in a mammal comprising administering to the mammal a compound having formula I wherein R₁ is heterocyclealkyl wherein the heterocycle of the heterocyclealkyl is selected from the group consisting of benzoisothiazolyl, isoquinolinyl, pyridinyl, quinolinyl, and tetrahydroquinolinyl, wherein the heterocycle is optionally substituted with 1 or 2 substituents independently selected from the group consisting of alkoxy, alkyl, and halogen; R₂ is selected from the group consisting of tert-butyl and 1,1-dichloroethyl; R₃ is heterocyclealkyl wherein the heterocycle of the heterocyclealkyl is selected from the group consisting of pyridinyl, quinolinyl, and thienyl; and R₄, R₅, and R₆ are as defined in formula I.

In another embodiment, the present invention discloses a method for treating neuropathic pain in a mammal comprising administering to the mammal a compound having formula I wherein R₁ is heterocyclealkyl; R₃ is heterocycle; and R₂, R₄, R₅, and R₆ are as defined in formula I.

In another embodiment, the present invention discloses a method for treating neuropathic pain in a mammal comprising administering to the mammal a compound having formula I wherein R₁ is heterocyclealkyl wherein the heterocycle of the heterocyclealkyl is selected from the group consisting of benzoisothiazolyl, isoquinolinyl, pyridinyl, quinolinyl, and tetrahydroquinolinyl, wherein the heterocycle is optionally substituted with 1 or 2 substituents independently selected from the group consisting of alkoxy, alkyl, and halogen; R₂ is selected from the group consisting of tert-butyl and 1,1-dichloroethyl; R₃ is heterocycle wherein the heterocycle is selected from the group consisting of pyridinyl, quinolinyl, and thienyl; and R₄, R₅, and R₆ are as defined in formula I.

In another embodiment, the present invention discloses a method for treating neuropathic pain in a mammal comprising administering to the mammal a compound having formula I wherein R₁ is arylalkyl; R₃ is alkyl; and R₂, R₄, R₅, and R₆ are as defined in formula I.

In another embodiment, the present invention discloses a method for treating neuropathic pain in a mammal comprising administering to the mammal a compound having formula I wherein R₁ is arylalkyl wherein the aryl is selected from the group consisting of naphthyl and phenyl, wherein the phenyl is optionally substituted with 1 or 2 substituents independently selected from the group consisting of alkoxy, alkyl, alkylthio, cyano, ethylenedioxy, halogen, methylenedioxy, and R_{A}R_{B}N—; R₂ is selected from the group consisting of tert-butyl and 1,1-dichloroethyl; R₃ is alkyl; and R_{A}, R_{B}, R₄, R₅, and R₆ are as defined in formula I.

In another embodiment, the present invention discloses a method for treating neuropathic pain in a mammal comprising administering to the mammal a compound having formula I wherein R₁ is arylalkyl; R₃ is heterocycle; and R₂, R₄, R₅, and R₆ are as defined in formula I.

In another embodiment, the present invention discloses a method for treating neuropathic pain in a mammal comprising administering to the mammal a compound having formula I wherein R₁ is arylalkyl wherein the aryl is selected from the group consisting of naphthyl and phenyl, wherein the phenyl is optionally substituted with 1 or 2 substituents independently selected from the group consisting of alkoxy, alkyl, alkylthio, cyano, ethylenedioxy, halogen, methylenedioxy, and R_{A}R_{B}N—; R₂ is selected from the group consisting of tert-butyl and 1,1-dichloroethyl; R₃ is heterocycle wherein the heterocycle is selected from the group consisting of quinolinyl, pyridinyl, and thienyl; and R_{A}, R_{B}, R₄, R₅, and R₆ are as defined in formula I.

In another embodiment, the present invention discloses a method for treating neuropathic pain in a mammal comprising administering to the mammal a compound having formula I wherein R₁ is arylalkyl; R₃ is heterocyclealkyl; and R₂, R₄, R₅, and R₆ are as defined in formula I.

In another embodiment, the present invention discloses a method for treating neuropathic pain in a mammal comprising administering to the mammal a compound having formula I wherein R₁ is arylalkyl wherein the aryl is selected from the group consisting of naphthyl and phenyl, wherein the phenyl is optionally substituted with 1 or 2 substituents independently selected from the group consisting of alkoxy, alkyl, alkylthio, cyano, ethylenedioxy, halogen, methylenedioxy, and R_{A}R_{B}N—; R₂ is selected from the group consisting of tert-butyl and 1,1-dichloroethyl; R₃ is heterocyclealkyl wherein the heterocycle of the heterocyclealkyl is selected from the group consisting of quinolinyl, pyridinyl, and thienyl; and R_{A}, R_{B}, R₄, R₅, and R₆ are as defined in formula I.

In another embodiment the present invention relates to a pharmaceutical composition comprising compounds of the present invention in combination with a pharmaceutically acceptable carrier.

### Methods for Preparing Compounds of the Invention

The compounds and processes of the present invention will be better understood in connection with the following synthetic schemes and methods, which illustrate a means by which the compounds of the invention can be prepared.

The compounds of this invention may be prepared by a variety of synthetic routes. Representative procedures are shown in Schemes 1-3.

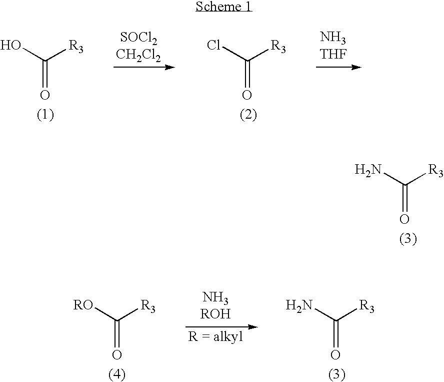

Amides of general formula (3), wherein R₃ is as defined in formula I, can be prepared as described in Scheme 1. Acids of general formula (1), purchased commercially or prepared using methods known to those of ordinary skill in the art, can be treated with thionyl chloride to provide acid chlorides of general formula (2). Acid chlorides of general formula (2) can be treated with ammonia to provide amides of general formula (3).

Additionally, esters of general formula (4) purchased commercially or prepared using methods known to those of ordinary skill in the art, can be treated with ammonia to provide amides of general formula (3).

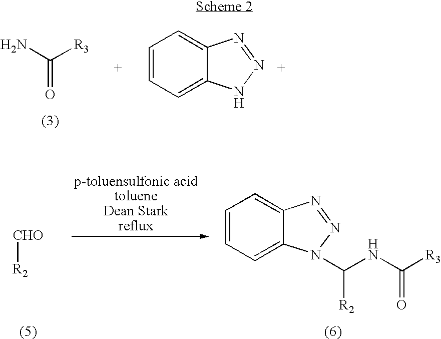

Benzotriazoles of general formula (6), wherein R₂ and R₃ are as defined in formula (I), can be prepared as described in Scheme 2. Amides of general formula (3) can be treated with benzotriazole and aldehydes of general formula (5), purchased commercially or prepared using methods known to those of ordinary skill in the art, to provide benzotriazoles of general formula (6).

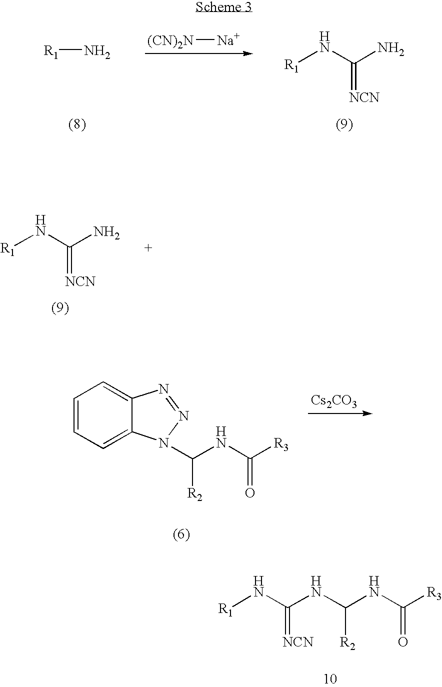

Compounds of general formula (10), wherein R₁, R₂, and R₃ are as defined in formula I, can be prepared as described in Scheme 3. Amines of general formula (8) purchased commercially or prepared using methods known to those of ordinary skill in the art, can be treated with sodium dicyanamide to provide cyanoguanidines of general formula (9). Cyanoguanidines of general formula (9) can be treated with benzotriazoles of general formula (6) and a base in a solvent including, but not limited to acetonitrile, N,N-dimethylformamide, or a combination thereof, to provide compounds of general formula (10).

### Compositions of the Invention

The present invention provides pharmaceutical compositions, which comprise compounds of the present invention formulated together with one or more non-toxic pharmaceutically acceptable carriers. The pharmaceutical compositions can be formulated for oral administration in solid or liquid form, for parenteral injection or for rectal administration.

The term “pharmaceutically acceptable carrier,” as used herein, means a non-toxic, inert solid, semi-solid or liquid filler, diluent, encapsulating material or formulation auxiliary of any type. Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The pharmaceutical compositions of this invention can be administered to humans and other mammals orally, rectally, parenterally, intracisternally, intravaginally, intraperitoneally, topically (as by powders, ointments or drops), bucally or as an oral or nasal spray. The term “parenterally,” as used herein, refers to modes of administration that include intravenous, intramuscular, intraperitoneal, intrasternal, subcutaneous, intraarticular injection and infusion.

Pharmaceutical compositions of this invention for parenteral injection comprise pharmaceutically acceptable sterile aqueous or nonaqueous solutions, dispersions, suspensions or emulsions and sterile powders for reconstitution into sterile injectable solutions or dispersions. Examples of suitable aqueous and nonaqueous carriers, diluents, solvents or vehicles include water, ethanol, polyols (propylene glycol, polyethylene glycol, glycerol, and the like), suitable mixtures thereof, vegetable oils (such as olive oil) and injectable organic esters such as ethyl oleate. Proper fluidity may be maintained, for example, by the use of a coating such as lecithin, by the maintenance of the required particle size in the case of dispersions, and by the use of surfactants.

These compositions may also contain adjuvants such as preservative agents, wetting agents, emulsifying agents, and dispersing agents. Prevention of the action of microorganisms may be ensured by various antibacterial and antifungal agents, for example, parabens, chlorobutanol, phenol, sorbic acid, and the like. It may also be desirable to include isotonic agents, for example, sugars, sodium chloride and the like. Prolonged absorption of the injectable pharmaceutical form may be brought about by the use of agents delaying absorption, for example, aluminum monostearate and gelatin. In some cases, in order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle. Suspensions, in addition to the active compounds, may contain suspending agents, as, for example, ethoxylated isostearyl alcohols, polyoxyethylene sorbitol and sorbitan esters, microcrystalline cellulose, aluminum metahydroxide, bentonite, agar-agar, tragacanth, and mixtures thereof.

If desired, and for more effective distribution, the compounds of the present invention can be incorporated into slow-release or targeted-delivery systems such as polymer matrices, liposomes, and microspheres. They may be sterilized, for example, by filtration through a bacteria-retaining filter or by incorporation of sterilizing agents in the form of sterile solid compositions, which may be dissolved in sterile water or some other sterile injectable medium immediately before use.

The active compounds can also be in micro-encapsulated form, if appropriate, with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound can be admixed with at least one inert diluent such as sucrose, lactose, or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of such composition that they release the active ingredient(s) only, or preferentially, in a certain part of the intestinal tract in a delayed manner. Examples of embedding compositions that can be used include polymeric substances and waxes.

Injectable depot forms are made by forming microencapsulated matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides) Depot injectable formulations are also prepared by entrapping the drug in liposomes or microemulsions which are compatible with body tissues.

The injectable formulations can be sterilized, for example, by filtration through a bacterial-retaining filter or by incorporating sterilizing agents in the form of sterile solid compositions which can be dissolved or dispersed in sterile water or other sterile injectable medium just prior to use.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic, parenterally acceptable diluent or solvent such as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid; b) binders such as carboxymethylcellulose, alginates, gelatin, polyvinylpyrrolidinone, sucrose, and acacia; c) humectants such as glycerol; d) disintegrating agents such as agar-agar, calcium carbonate, potato or tapioca starch, alginic acid, certain silicates, and sodium carbonate; e) solution retarding agents such as paraffin); f) absorption accelerators such as quaternary ammonium compounds; g) wetting agents such as cetyl alcohol and glycerol monostearate; h) absorbents such as kaolin and bentonite clay; and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract in a delayed manner. Examples of embedding compositions that can be used include polymeric substances and waxes.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, eye ointments, powders and solutions are also contemplated as being within the scope of this invention.

The ointments, pastes, creams and gels may contain, in addition to an active compound of this invention, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the compounds of this invention, excipients such as lactose, talc, silicic acid, aluminum hydroxide, calcium silicates and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

Compounds of the present invention may also be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming liposomes may be used. The present compositions in liposome form may contain, in addition to the compounds of the present invention, stabilizers, preservatives, excipients, and the like. The preferred lipids are the natural and synthetic phospholipids and phosphatidylcholines (lecithins) used separately or together. Methods to form liposomes are known in the art. See, for example, Prescott, Ed., Methods in Cell Biology, Volume XIV, Academic Press, New York, N.Y., (1976), p 33 et seq.

The terms “pharmaceutically acceptable salts, esters and amides,” as used herein, refer to carboxylate salts, amino acid addition salts, zwitterions, esters and amides of compounds of formula I which are, within the scope of sound medical judgment, suitable for use in contact with the tissues of humans and lower animals without undue toxicity, irritation, allergic response, and the like, are commensurate with a reasonable benefit/risk ratio, and are effective for their intended use.

The term “pharmaceutically acceptable salt,” as used herein, refers to salts that are well known in the art. For example, S. M Berge et al. describe pharmaceutically acceptable salts in detail in J. Pharmaceutical Sciences, Vol. 66, pages 1-19 (1977). Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid, or malonic acid or by using other methods used in the art such as ion exchange. Other pharmaceutically acceptable salts include nitrate, bisulfate, borate, formate, butyrate, valerate, 3-phenylpropionate, camphorate, adipate, benzoate, oleate, palmitate, stearate, laurate, lactate, fumarate, ascorbate, aspartate, nicotinate, p-toluenesulfonate, camphorsulfonate, methanesulfonate, 2-hydroxyethanesulfonate, gluconate, glucoheptonate, lactobionate, glycerophosphate, pectinate, lauryl sulfate, and the like, metal salts such as sodium, potassium, magnesium or calcium salts or amino salts such as ammonium, triethylamine salts, and the like, all of which may be prepared according to conventional methods.

The term “pharmaceutically acceptable ester,” as used herein, refers to esters of compounds of the present invention which hydrolyze in vivo and include those that break down readily in the human body to leave the parent compound or a salt thereof. Examples of pharmaceutically acceptable, non-toxic esters of the present invention include C₁-to-C₆ alkyl esters and C₅-to-C₇ cycloalkyl esters, although C₁-to-C₄ alkyl esters are preferred.

The term “pharmaceutically acceptable amide,” as used herein, refers to non-toxic amides of the present invention derived from ammonia, primary C₁-to-C₆ alkyl amines and secondary C₁-to-C₆ dialkyl amines. In the case of secondary amines, the amine may also be in the form of a 5- or 6-membered heterocycle containing one nitrogen atom. Amides derived from ammonia, C₁-to-C₃ alkyl primary amides and C₁-to-C₂ dialkyl secondary amides are preferred. Amides of the compounds of formula I may be prepared according to conventional methods. It is intended that amides of the present invention include amino acid and peptide derivatives of the compounds of formula I, as well.

The term “pharmaceutically acceptable prodrug” or “prodrug,” as used herein, represents those prodrugs of the compounds of the present invention which are, within the scope of sound medical judgment, suitable for use in contact with the tissues of humans and lower animals without undue toxicity, irritation, allergic response, and the like, commensurate with a reasonable benefit/risk ratio, and effective for their intended use. Prodrugs of the present invention may be rapidly transformed in vivo to the parent compound of the above formula, for example, by hydrolysis in blood. A thorough discussion is provided in T. Higuchi and V. Stella, “Pro-drugs as Novel Delivery Systems” V. 14 of the A.C.S. Symposium Series, and in Edward B. Roche, ed., “Bioreversible Carriers in Drug Design”, American Pharmaceutical Association and Pergamon Press (1987), hereby incorporated by reference.

The compounds of the invention, including but not limited to those specified in the examples, possess potassium channel opening activity in mammals (especially humans). As potassium channel openers, the compounds of the present invention are useful for the treatment and prevention of neuropathic pain.

When used in the above or other treatments, a therapeutically effective amount of one of the compounds of the present invention can be employed in pure form or, where such forms exist, in pharmaceutically acceptable salt, ester, amide or prodrug form. Alternatively, the compound can be administered as a pharmaceutical composition containing the compound of interest in combination with one or more pharmaceutically acceptable excipients. The phrase “therapeutically effective amount” of the compound of the invention means a sufficient amount of the compound to treat disorders, at a reasonable benefit/risk ratio applicable to any medical treatment. It will be understood, however, that the total daily usage of the compounds and compositions of the present invention will be decided by the attending physician within the scope of sound medical judgment. The specific therapeutically effective dose level for any particular patient will depend upon a variety of factors including the disorder being treated and the severity of the disorder; activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts. For example, it is well within the skill of the art to start doses of the compound at levels lower than required to achieve the desired therapeutic effect and to gradually increase the dosage until the desired effect is achieved.

The total daily dose of the compounds of this invention administered to a human or lower animal may range from about 0.003 to about 30 mg/kg/day. For purposes of oral administration, more preferable doses can be in the range of from about 0.01 to about 10 mg/kg/day. If desired, the effective daily dose can be divided into multiple doses for purposes of administration; consequently, single dose compositions may contain such amounts or submultiples thereof to make up the daily dose.

### EXAMPLES

### Example 1

### N-[1-({(cyanoimino)[(2-methyl-3-pyridinyl)amino]methyl}amino)-2,2-dimethylpropyl]-2-(3,4-difluorophenyl)acetamide

### Example 1A

### 2-(3,4-difluorophenyl)acetamide

To a solution of 2-(3,4-difluorophenyl)acetic acid (5.1 g, 30.33 mmol) in 50 mL of anhydrous dichloromethane was added SOCl₂ (4.33 g, 36.4 mmol), and a drop of dimethylformamide as catalyst. The mixture was stirred at room temperature for 2 hr, solvent and excess SOCl₂ were removed under reduced pressure. The crude product was dissolved in 50 mL of THF, cooled to 0° C. and liquid ammonia was added through condenser dropwise for 20 minutes. The reaction mixture was concentrated, the product precipitated with 30 ml of water, filtered, and dried to afford 4.55 g of 2-(3,4-difluorophenyl)acetamide as white crystalline solid. MS (ESI⁺) m/z 172 (M+H)⁺.

### Example 1B

### N-(1-benzotriazol-1-yl-2,2-dimethylpropyl)-2-(3,4-difluorophenyl)acetamide

A suspension of example 1A (3.9 g, 22.8 mmol), trimethylacetaldehyde (4.15 g, 48.2 mmol), and 1H-1,2,3-benzotriazole (2.72 g, 22.8 mmol) in toluene (75 mL) was treated with p-toluenesulfonic acid (0.217 g, 1.14 mmol). The solution was heated at reflux under Dean-Stark conditions for 10 hours, cooled gradually to ambient temperature, and further cooled at 5° C. The white precipitate was collected by filtration and washed with 50% ether/hexanes (100 mL) to provide 4.44 g of the desired product as a white solid.

MS (ESI⁺) m/z 359 (M+H)⁺.

### Example 1C

### N″-cyano-N-(2-methyl-3-pyridinyl)guanidine

To a solution of 2-methyl-3-pyridinylamine (22.7 g, 0.21 moles) in water was added 6N HCl (42 mL, 0.25 moles), and sodium dicyanamide (22.46 g, 0.25 moles). The reaction was stirred overnight at 60° C. The reaction was continued for an additional 36 hours with one more equivalent of 6N HCl and sodium dicyanamide added at the 12 and 24 hr time points. The reaction mixture was cooled, the precipitate collected, and washed with water. The crude product was purified by Soxhlet extraction method using 5% methanol in methylene chloride, followed by flash chromatography (sequential elution with 2, 5, and 10% of methanol in methylene chloride) to provide the title compound. MS (ESI⁺) m/z 176 (M+H)⁺.

### Example 1D

### N-[1-({(cyanoimino)[(2-methyl-3-pyridinyl)amino]methyl}amino)-2,2-dimethylpropyl]-2-(3,4-difluorophenyl)acetamide

A solution of example 1B (204 mg, 0.57 mmol) and example 1C (100 mg, 0.57 mmol) in DMF or CH₃CN (4 mL) at 23° C. was treated with finely powdered Cs₂CO₃ (465 mg, 1.43 mmol). The reaction mixture was stirred for 10 hours, then partitioned between ethyl acetate (15 mL) and water (10 mL). The aqueous layer was extracted with ethyl acetate (10 mL), and the combined organics were washed with water (2×5 mL) and brine (5 mL). The organic portions were dried (Na₂SO₄), filtered, and concentrated. Purification by flash chromatography (elution with 1% methanol/CH₂Cl₂) provided 106 mg of the title compound as a white solid. mp 58-60° C.; MS (ESI⁺) m/z 415 (M+H)⁺; ¹H NMR (300 MHz, DMSO-d₆) δ 0.91 (s, 9 H), 2.32 (s, 3 H), 3.51 (d, J=14.2 Hz, 1 H), 3.56 (d, J=14.6 Hz, 1 H), 5.42 (dd, J=8.8, 8.8 Hz, 1 H), 6.68 (br m, 1 H), 7.11 (br m, 1 H), 7.23 (dd, J=8.1, 4.8 Hz, 1 H), 7.35 (m, 3 H), 7.47 (dd, J=8.0, 1.5 Hz, 1 H), 8.20 (d, J=8.5 Hz, 1 H), 8.34 (dd, J=4.8, 1.4 Hz, 1 H); Anal. calcd. for C₂₁H₂₄F₂N₆O: C, 60.86; H, 5.84; N, 20.28. Found: C, 61.01; H, 5.62; N, 20.06.

### Example 2

### 2-(4-chlorophenyl)-N-[1-({(cyanoimino)[(2-methyl-3-pyridinyl)amino]methyl}amino)-2,2-dimethylpropyl]acetamide

### Example 2A

### N-[1-(1H-1,2,3-benzotriazol-1-yl)-2,2-dimethylpropyl]-2-(4-chlorophenyl)acetamide

1H-1,2,3-Benzotriazole, trimethylacetaldehyde and 2-(4-chlorophenyl)acetamide, (prepared as described in El-Rayyes, N. R.; Al-Hajjar, F. H. J. Heterocycl. Chem., Vol. 21 pages1473-1477 (1984)), were processed as described in example 1B to provide the title compound. MS (ESI⁺) m/z 357 (M+H)⁺.

### Example 2B

### 2-(4-chlorophenyl)-N-[1-({(cyanoimino)[(2-methyl-3-pyridinyl)amino]methyl}amino)-2,2-dimethylpropyl]acetamide

Examples 1C and 2A were processed as described in example 1D to provide the title compound. mp 180-181° C.; MS (ESI⁺) m/z 413 (M+H)⁺; ¹H NMR (300 MHz, DMSO-d₆) δ 0.91 (s, 9 H), 2.32 (s, 3 H), 3.50 (d, J=14.2 Hz, 1 H), 3.56 (d, J=14.2 Hz, 1 H), 5.42 (dd, J=8.8, 8.8 Hz, 1 H), 6.68 (d, J=9.2 Hz, 1 H), 7.31 (m, 5 H), 7.47 (dd, J=8.1, 1.4 Hz, 1 H), 8.21 (d, J=8.1 Hz, 1 H), 8.34 (dd, J=4.8, 1.4 Hz, 1 H), 9.09 (br s, 1 H); Anal. calcd. for C₂₁H₂₅ClN₆O: C, 61.08; H, 6.10; N, 20.35. Found: C, 60.80; H, 5.93; N, 20.14.

### Example 3

### N-[1-({(cyanoimino)[(2-methyl-3-pyridinyl)amino]methyl}amino)-2,2-dimethylpropyl]-4-(4-methoxyphenyl)butanamide

### Example 3A

### N-(1-benzotriazol-1-yl-2,2-dimethylpropyl)-4-(4-methoxyphenyl)butyramide

1H-1,2,3-Benzotriazole, trimethylacetaldehyde and 4-(4-methoxyphenyl)butanamide, (prepared as described in Ganellin, et al., Arch. Pharm. Vol. 331 (12), pages 395-404 (1998)), were processed as described in example 1B to provide the title compound. MS (ESI⁺) m/z 381 (M+H)⁺.

### Example 3B

### N-[1-({(cyanoimino)[(2-methyl-3-pyridinyl)amino]methyl}amino)-2,2-dimethylpropyl]-4-(4-methoxyphenyl)butanamide

Example 1C and 3A were processed as described in example 1D to provide the title compound. mp 65-68° C.; MS (ESI⁺) m/z 437 (M+H)⁺; ¹H NMR (300 MHz, DMSO-d₆) δ 0.92 (s, 9 H), 1.77 (m, 2 H), 2.17 (t, J=8.0 Hz, 2 H), 2.38 (s, 3 H), 2.49 (m, 2 H), 3.71 (s, 3 H), 5.43 (dd, J=8.8, 8.8 Hz, 1 H), 6.63 (br m, 1 H), 6.84 (m, 2 H), 7.09 (m, 2 H), 7.24 (dd, J=8.0, 4.9 Hz, 1 H), 7.51 (dd, J=8.0, 1.5 Hz, 1 H), 7.99 (br d., J=9.2, 1 H), 8.34 (dd, J=4.8, 1.4 Hz, 1 H), 9.23 (br s, 1 H); Anal. calcd. for C₂₄H₃₂N₆O₂0.3H₂O: C, 65.22; H, 7.43; N, 19.02. Found: C, 65.15; H, 7.34; N, 18.83.

### Example 4

### N-[1-({(cyanoimino)[(2-methyl-3-pyridinyl)amino]methyl}amino)-2,2-dimethylpropyl]-2-(3-fluoro-4-methylphenyl)acetamide

### Example 4A

### 2-(3-fluoro-4-methylphenyl)acetamide

2-(3-Fluoro-4-methylphenyl)acetic acid, SOCl₂, and liquid NH₃ were processed as described in example 1A to provide the title compound. MS (ESI⁺) m/z 168 (M+H)⁺.

### Example 4B

### N-[1-(1H-1,2,3-benzotriazol-1-yl)-2,2-dimethylpropyl]-2-(3-fluoro-4-methylphenyl)acetamide

1H-1,2,3-Benzotriazole, trimethylacetaldehyde and 2-(3-fluoro-4-methylphenyl)acetamide were processed as described in example 1B to provide the title compound.

MS (ESI⁺) m/z 355 (M+H)⁺.

### Example 4C

### N-[1-({(cyanoimino)[(2-methyl-3-pyridinyl)amino]methyl}amino)-2,2-dimethylpropyl]-2-(3-fluoro-4-methylphenyl)acetamide

Examples 1C and 4B were processed as described in example 1D to provide the title compound. mp 75-77° C.; MS (ESI⁺) m/z 411 (M+H)⁺; ¹H NMR (300 MHz, DMSO-d₆) δ 0.91 (s, 9 H), 2.20 (d, J=1.7 Hz, 3 H), 2.33 (s, 3 H), 3.47 (d, J=14.6 Hz, 1 H), 3.53 (m, J=13.9 Hz, 1 H), 5.42 (dd, J=9.0, 9.0 Hz, 1 H), 6.69 (d, J=8.5 Hz, 1 H), 7.06 (m, 2 H), 7.22 (m, 2 H), 7.47 (dd, J=8.1, 1.7 Hz, 1 H), 8.18 (d, J=7.8 Hz 1 H), 8.34 (dd, J=4.8, 1.4 Hz, 1 H), 9.09 (br s, 1 H); Anal. calcd. for C₂₂H₂₇FN₆O 0.1 CF₃COOH: C, 63.20; H, 6.47; N, 19.92. Found: C, 63.13; H, 6.09; N, 19.90.

### Example 5

### N-[1-({(cyanoimino)[(2-methyl-3-pyridinyl)amino]methyl}amino)-2,2-dimethylpropyl]-5-phenylpentanamide

### Example 5A

### N-[1-(1H-1,2,3-benzotriazol-1-yl)-2,2-dimethylpropyl]-5-phenylpentanamide

1H-1,2,3-Benzotriazole, trimethylacetaldehyde and 5-phenylpentanamide, (prepared as described in De Tar; Carmack; J. Amer. Chem. Soc. Vol 68, pages 2025-2028 (1946)), were processed as described in example 1B to provide the title compound. MS (ESI⁺) m/z 365 (M+H)⁺.

### Example 5B

### N-[1-({(cyanoimino)[(2-methyl-3-pyridinyl)amino]methyl}amino)-2,2-dimethylpropyl]-5-phenylpentanamide

Example 1C and 5A were processed as described in example 1D to provide the title compound. mp 133-134° C.; MS (ESI⁺) m/z 421 (M+H)⁺; ¹H NMR (300 MHz, DMSO-d₆) δ 0.92 (s, 9 H), 1.55 (m, 4 H), 2.21 (m, 2 H), 2.36 (s, 3 H), 2.50 (m, 2 H), 2.58 (t, J=7.1 Hz, 2 H), 5.42 (dd, J=9.0, 9.0 Hz, 1 H), 7.21 (m, 7 H), 7.48 (d, J=7.8 Hz, 1 H), 8.01 (br d, J=7.8 Hz, 1 H 1 H), 8.34 (br m, 1 H), 9.24 (br s, 1 H); Anal. calcd. for C₂₄H₃₂N₆O0.2 H₂O: C, 67.96; H, 7.70; N, 19.81. Found: C, 67.82; H, 8.01; N, 19.80.

### Example 6

### N-[1-({(cyanoimino)[(2-methyl-3-pyridinyl)amino]methyl}amino)-2,2-dimethylpropyl]-2-(4-fluorophenyl)acetamide

### Example 6A

### N-[1-(1H-1,2,3-benzotriazol-1-yl)-2,2-dimethylpropyl]-2-(4-fluorophenyl)acetamide

1H-1,2,3-Benzotriazole, trimethylacetaldehyde and 2-(4-fluorophenyl)acetamide were processed as described in example 1B to provide the title compound.

MS (ESI⁺) m/z 341 (M+H)⁺.

### Example 6B

### N-[1-({(cyanoimino)[(2-methyl-3-pyridinyl)amino]methyl}amino)-2,2-dimethylpropyl]-2-(4-fluorophenyl)acetamide

Example 1C and 6A were processed as described in example 1D to provide the title compound. mp 166-167° C.; MS (ESI⁺) m/z 397 (M+H)⁺; ¹H NMR (300 MHz, DMSO-d₆) δ 0.91 (s, 9 H), 2.32 (s, 3 H), 3.48 (d, J=14.2 Hz, 1 H), 3.55 (d, J=14.2 Hz, 1 H), 5.41 (dd, J=9.0, 9.0 Hz, 1 H), 6.69 (d, J=8.1 Hz, 1 H), 7.13 (m, 2 H), 7.23 (dd, J=7.8, 4.8 Hz, 1 H), 7.31 (m, 2 H), 7.47 (dd, J=8.0, 1.5 Hz, 1 H), 8.19 (d, J=8.1, Hz 1 H), 8.34 (dd, J=4.6, 1.2 Hz, 1 H), 9.10 (br s, 1 H); Anal. calcd. for C₂₁H₂₅FN₆O: C, 63.62; H, 6.36; N, 21.20. Found: C, 63.44; H, 6.60; N, 21.10.

### Example 7

### N-[1-({(cyanoimino)[(2-methyl-3-pyridinyl)amino]methyl}amino)-2,2-dimethylpropyl]-4-(2-thienyl)butanamide

### Example 7A

### N-[1-(1H-1,2,3-benzotriazol-1-yl)-2,2-dimethylpropyl]-4-(2-thienyl)butanamide

1H-1,2,3-Benzotriazole, trimethylacetaldehyde and 4-(2-thienyl)butanamide, (prepared as described in Blanchette; Brown; J. Amer. Chem. Soc. Vol 74, page 1066 (1952)), were processed as described in example 1B to provide the title compound. MS (ESI⁺) m/z 357 (M+H)⁺.

### Example 7B

### N-[1-({(cyanoimino)[(2-methyl-3-pyridinyl)amino]methyl}amino)-2,2-dimethylpropyl]-4-(2-thienyl)butanamide

Example 1C and 7A were processed as described in example 1D to provide the title compound. mp 66-68° C.; MS (ESI⁺) m/z 413 (M+H)⁺;¹H NMR (300 MHz, DMSO-d₆) δ 0.92 (s, 9 H), 1.85 (m, 2 H), 2.23 (t, J=7.1 Hz, 2 H), 2.38 (s, 3 H), 2.79 (t, J=7.5 Hz, 2 H), 5.44 (dd, J=9.0, 9.0 Hz, 1 H), 6.60 (br m., 1 H), 6.84 (m, 1 H), 6.94 (dd, J=5.1, 3.4 Hz, 1 H), 7.24 (dd, J=8.0, 4.6 Hz, 1 H), 7.31 (dd, J=5.1, 1.4 Hz, 1 H), 7.51 (dd, J=8.0, 1.2 Hz, 1 H), 8.02 (br d, J=7.5 Hz, 1 H), 8.33 (br d, J=4.75 Hz, 1 H), 9.19 (br s, 1 H); Anal. calcd. for C₂₁H₂₈FN₆OS0.3 H₂O: C, 60.35; H, 6.90; N, 20.11. Found: C, 60.16; H, 6.78; N, 20.07.

### Example 8

### N-[1-({(cyanoimino)[(2-methyl-3-pyridinyl)amino]methyl}amino)-2,2-dimethylpropyl]-2-[4-(methylthio)phenyl]acetamide

### Example 8A

### N-[1-(1H-1,2,3-benzotriazol-1-yl)-2,2-dimethylpropyl]-2-[4-(methylthio)phenyl]acetamide

1H-1,2,3-Benzotriazole, trimethylacetaldehyde and 2-[4-(methylthio)phenyl]acetamide, (prepared as described in Ruechardt; Boeck; Chem. Ber. Vol 100 page 654 (1967)), were processed as described in example 1B to provide the title compound. MS (ESI⁺) m/z 369 (M+H)⁺.

### Example 8B

### N-[1-({(cyanoimino)[(2-methyl-3-pyridinyl)amino]methyl}amino)-2,2-dimethylpropyl]-2-8 4-(methylthio)phenyl]acetamide

Example 1C and 8A were processed as described in example 1D to provide the title compound. mp 172-173° C.; MS (ESI⁺) m/z 425 (M+H)⁺; ¹H NMR (300 MHz, DMSO-d₆) δ 0.91 (s, 9 H), 2.33 (s, 3 H), 2.45 (s, 3 H), 3.45 (d, J=14.2 Hz, 1 H), 3.52 (d, J=13.9 Hz, 1 H), 5.42 (dd, J=9.0, 9.0 Hz, 1 H), 6.69 (d, J=8.8 Hz, 1 H), 7.21 (s, 4 H), 7.24 (dd, J=9.0, 5.1 Hz, 1 H), 7.46 (dd, J=8.0, 1.5 Hz, 1 H), 8.17 (d, J=7.5 Hz, 1 H), 8.34 (dd, J=4.8, 1.4 Hz, 1 H), 9.11 (s, 1 H); Anal. calcd. for C₂₂H₂₈N₆OS: C, 62.24; H, 6.65; N, 19.79. Found: C, 61.98; H, 6.75; N, 19.56.

### Example 9

### 2-(4-chlorophenyl)-N-[1-({(cyanoimino)[(2-ethyl-3-pyridinyl)amino]methyl}amino)-2,2-dimethylpropyl]acetamide

### Example 9A

### 2-ethyl-3-pyridinamine

To a solution of 2-vinyl-3-nitropyridine (0.7 g, 4.7 mmoles), (prepared as described in Jun Li et al., Tetrahedron, pages 393-400 (1998)), in methanol (25 ml) was added 70 mg of 10% palladium on charcoal. The flask was evacuated and supplied with hydrogen gas from a balloon for overnight. The reaction mixture was filtered through celite and dried to provide 0.51 g of the title compound. MS (ESI⁺) m/z 123 (M+H)⁺.

### Example 9B

### N″-cyano-N-(2-ethyl-3-pyridinyl)guanidine

Example 9A was processed as described in example 1C to provide the title compound. MS (ESI⁺) m/z 190 (M+H)⁺.

### Example 9C

### 2-(4-chlorophenyl)-N-[1-({(cyanoimino)[(2-ethyl-3-pyridinyl)amino]methyl}amino)-2,2-dimethylpropyl]acetamide

Example 9B and 2A were processed as described in example 1D to provide the title compound.mp 112-114° C.; MS (ESI⁺) m/z 427 (M+H)⁺; ¹NMR (300 MHz, DMSO-d₆) δ 0.91 (s, 9 H), 1.14 (t, J=7.63 Hz, 3 H), 2.65 (q, J=7.5 Hz, 2 H), 3.49 (d, J=14.2 Hz, 1 H), 3.55 (d, J=14.2 Hz, 1 H), 5.42 (dd, J=8.8, 8.8 Hz, 1 H), 6.63 (br d, J=9.8 Hz, 1 H), 7.31 (m, 5 H), 7.46 (dd, J=7.8, 1.7 Hz, 1 H), 8.21 (br d, J=8.5 Hz, 1 H), 8.41 (m, 1 H), 9.08 (br s, 1 H); Anal. calcd. for C₂₂H₂₇ClN₆O0.4 CH₃OH: C, 61.24; H, 6.47; N, 19.13. Found: C, 61.16; H, 6.32; N, 18.76.

### Example 10

### N-[1-({(cyanoimino)[(2-methylphenyl)amino]methyl}amino)-2,2-dimethylpropyl]-4-(4-methoxyphenyl)butanamide

N″-Cyano-(2-methylphenyl)guanidine and example 3A were processed as described in example 1D to provide the title compound. MS (ESI⁺) m/z 436 (M+H)⁺; ¹H NMR (500 MHz, MeOH-d₄) δ 0.95 (s, 9 H), 1.88 (m, 2 H), 2.24 (t, J=7.49 Hz, 2 H), 2.27 (s, 3 H), 2.57 (t, J=7.80 Hz, 2 H), 3.75 (s, 3 H), 5.47 (s, 1 H), 6.82 (d, J=8.4 Hz, 2 H), 7.09 (d, J=8.73 Hz, 2 H), 7.19 (m, 1 H), 7.25 (m, 2 H), 7.31 (m, 1 H).

### Example 11

### N-[1-({(cyanoimino)[(2-methylphenyl)amino]methyl}amino)-2,2-dimethylpropyl]-2-(3,4-dimethoxyphenyl)acetamide

### Example 11A

### N-[1-(1H-1,2,3-benzotriazol-1-yl)-2,2-dimethylpropyl]-2-(3,4-dimethoxyphenyl)acetamide

1H-1,2,3-Benzotriazole, trimethylacetaldehyde and 2-(3,4-dimethoxyphenyl)acetamide were processed as described in example 1B to provide the title compound. MS (ESI⁺) m/z 383 (M+H)⁺.

### Example 11B

### N-[1-({(cyanoimino)[(2-methylphenyl)amino]methyl}amino)-2,2-dimethylpropyl]-2-(3,4-dimethoxyphenyl)acetamide

N″-Cyano-N-(2-methylphenyl)guanidine, (prepared as described in Curd et al., J. Chem. Soc. pages 1630-1634 (1948)), and example 11A were processed as described in example 1D to provide the title compound. mp 156-157° C.; MS (ESI⁺) m/z 438 (M+H)⁺; ¹H NMR (300 MHz, DMSO-d₆) δ 0.88 (s, 9 H), 2.14 (s, 3 H), 3.39 (d, J=5.8 Hz, 1 H), 3.43 (d, J=5.8 Hz, 1 H), 3.72 (s, 6 H), 5.42 (dd, J=9.2 Hz, 1 H), 6.26 (br s, 1 H), 6.74-6.79 (m, 1 H), 6.85-6.90 (m, 2 H), 7.05-7.10 (m, 1 H) 7.18-7.30 (m, 3 H), 8.15 (d, J=8.5 Hz, 1 H), 9.04 (s, 1 H); Anal. calcd. for C₂₄H₃₁N₅O₃ 0.04H₂O: C, 65.88; H, 7.14; N, 16.01. Found: C, 64.53; H, 7.04; N, 15.50.

### Example 12

### (−) N-[1-({(cyanoimino)[(2-methylphenyl)amino]methyl}amino)-2,2-dimethylpropyl]-2-(3,4-dimethoxyphenyl)acetamide

Example 11B (2.4 g) was chromatographed over a Daicel Chiral Technologies Chiralcel AS chiral column (2.0×25 cm) eluting with 20% ethyl acetate/Hexanes (rate 10 ml/min) to provide 837 mg (retention time=14 minutes) of the title compound as the less polar enantiomer. mp 156-157° C.; [α]_{D}²³-3.64° (c 0.60, MeOH); MS (ESI⁺) m/z 438 (M+H)⁺; ¹H NMR (300 MHz, DMSO-d₆) δ 0.86 (s, 9 H), 2.14 (s, 3 H), 3.38 (d, J=5.8 Hz, 1 H), 3.44 (d, J=5.8 Hz, 1 H), 3.72 (s, 6 H), 5.42 (dd, J=9.2 Hz, 1 H), 6.27 (br s, 1 H), 6.75-6.78 (m, 1 H), 6.86-6.89 (m, 2 H), 7.05-7.08(m, 1 H), 7.18-7.30 (m, 3 H), 8.15 (d, J=8.5 Hz, 1 H), 9.04 (s, 1 H); Anal. calcd. for C₂₄H₃₁N₅O₃: C, 65.88; H, 7.14; N, 16.01. Found: C, 65.87; H, 7.04; N, 16.05.

### Example 13

### (+) N-[1-({(cyanoimino)[(2-methylphenyl)amino]methyl}amino)-2,2-dimethylpropyl]-2-(3,4-dimethoxyphenyl)acetamide

Example 11B (2.4 g) was chromatographed over a Daicel Chiral Technologies Chiralcel AS chiral column (2.0×25 cm) eluting with 20% ethyl acetate/Hexanes (rate 10 ml/minute) to provide 1.025 g (retention time=24 minutes) of the title compound as the more polar enantiomer. mp 156-157° C.; [α]_{D}²³+3.56°(c 0.95, MeOH); MS (ESI⁺) m/z 438 (M+H)⁺; ¹H NMR (300 MHz, DMSO-d₆) δ 0.88 (s, 9 H), 2.14 (s, 3 H), 3.39 (d, J=5.8 Hz, 1 H), 3.43 (d, J=5.8 Hz, 1 H), 3.72 (s, 6 H), 5.42 (dd, J=9.2 Hz, 1 H), 6.26 (br s, 1 H), 6.74-6.79 (m, 1 H), 6.85-6.90 (m, 2 H), 7.05-7.10 (m, 1 H) 7.18-7.30 (m, 3 H), 8.15 (d, J=8.5 Hz, 1 H), 9.04 (s, 1 H); Anal. calcd. for C₂₄H₃₁N₅O₃: C, 65.88; H, 7.14; N, 16.01. Found: C, 65.65; H, 7.19; N, 16.31.

### Example 14

### N-[1-({(cyanoimino)[(2-methylphenyl)amino]methyl}amino)-2,2-dimethylpropyl]-2-(4-methoxyphenyl)acetamide

### Example 14A

### N-[1-(1H-1,2,3-benzotriazol-1-yl)-2,2-dimethylpropyl]-2-(4-methoxyphenyl)acetamide

1H-1,2,3-Benzotriazole, trimethylacetaldehyde and 2-(4-methoxyphenyl)acetamide were processed as described in example 1B to provide the title compound. MS (ESI⁺) m/z 353 (M+H)⁺.

### Example 14B

### N-[1-({(cyanoimino)[(2-methylphenyl)amino]methyl}amino)-2,2-dimethylpropyl]-2-(4-methoxyphenyl)acetamide

N″-Cyano-N-(2-methylphenyl)guanidine and example 14A were processed as described in example 1D to provide the title compound. mp 174° C.; MS (ESI⁺) m/z 408 (M+H)⁺; ¹H NMR (300 MHz, DMSO-d₆) δ 0.86 (s, 9 H), 2.14 (s, 3 H), 3.38 (d, J=13.9 Hz, 1 H), 3.44 (d, J=14.2 Hz, 1 H), 3.72 (s, 3 H), 5.40 (dd, J=9.2, 9.2 Hz, 1 H), 6.24 (br s, 1 H), 6.84-6.89 (m, 2 H), 7.03-7.30 (m, 6 H), 8.15 (d, J=8.5 Hz, 1 H), 9.02 (m, 1 H); Anal. calcd. for C₂₃H₂₉N₅O₂: C, 67.79; H, 7.17; N, 17.19. Found: C, 67.65; H, 7.20; N, 17.36.

### Example 15

### 2-(1,3-benzodioxol-5-yl)-N-[1-({(cyanoimino)[(2-methylphenyl)amino]methyl}amino)-2,2-dimethylpropyl]acetamide

### Example 15A

### 2-(1,3-benzodioxol-5-yl)-N-[1-(1H-1,2,3-benzotriazol-1-yl)-2,2-dimethylpropyl]acetamide

1H-1,2,3-Benzotriazole, trimethylacetaldehyde and 2-(1,3-benzodioxol-5-yl)acetamide, (prepared as described in Kamochi, Yasuko; Watanabe, Yasuo; Heterocycles Vol. 26 (9), pages 2385-2391 (1987)), were processed as described in example 1B to provide the title compound. MS (ESI⁺) m/z 367 (M+H)⁺.

### Example 15B

### 2-(1,3-benzodioxol-5-yl)-N-[1-({(cyanoimino)[(2-methylphenyl)amino]methyl}amino)-2,2-dimethylpropyl]acetamide

N″-Cyano-N-(2-methylphenyl)guanidine and example 15A were processed as described in example 1D to provide the title compound. mp 152-153° C.; MS (ESI⁺) m/z 422 (M+H)⁺; ¹H NMR (300 MHz, DMSO-d₆) δ 0.87 (s, 9 H), 2.14 (s, 3 H), 3.34 (d, J=15.3 Hz, 1 H), 3.42 (d, J=15.3 Hz, 1 H), 5.41 (dd, J=9.2 Hz, 1 H), 5.98 (s, 2 H), 6.26 (br s, 1 H), 6.71 (dd, J=7.8, 1.7 Hz, 1 H), 6.88-6.86 (m, 2 H), 7.05-7.11 (m, 1 H), 7.17-7.31 (m, 3 H), 8.15 (d, J=8.8 Hz, 1 H), 9.02 (m, 1 H); Anal. calcd. for C₂₃H₂₇N₅O₃; C, 65.54; H, 6.46; N, 16.62. Found: C, 65.26; H, 6.37; N, 16.51.

### Example 16

### N-[1-({(cyanoimino)[(2-methylphenyl)amino]methyl}amino)-2,2-dimethylpropyl]-2-[4-(methylthio)phenyl]acetamide

N″-Cyano-N-(2-methylphenyl)guanidine and example 8A were processed as described in example 1D to provide the title compound. mp 151-152° C.; MS (ESI⁺) m/z 424 (M+H)⁺; ¹H NMR (300 MHz, DMSO-d₆) δ 0.87 (s, 9 H), 2.13 (s, 3 H), 3.42 (d, J=13.9 Hz, 1 H), 3.48 (d, J=13.9 Hz, 1 H), 5.41 (dd, J=8.8, 8.8 Hz, 1 H), 6.23 (br s, 1 H), 7.01-7.32(m, 8 H), 8.20 (d, J=8.8 Hz, 1 H), 9.01 (m, 1 H); Anal. calcd. for C₂₃H₂₉N₅OS0.5H₂O: C, 63.80; H, 6.70; N, 16.18. Found: C, 63.67; H, 7.04; N, 16.18.

### Example 17

### 2-(4-chlorophenyl)-N-[1-({(cyanoimino)[(2-methylphenyl)amino]methyl}amino)-2,2-dimethylpropyl]acetamide

N″-Cyano-N-(2-methylphenyl)guanidine and example 2A were processed as described in example 1D to provide the title compound. mp 181-182° C.; MS (ESI⁺) m/z 422 (M+H)⁺; ¹H NMR (300 MHz, DMSO-d₆) δ 0.86 (s, 9 H), 2.13 (s, 3 H), 3.46 (d, J=14.2 Hz, 1 H), 3.53 (d, J=14.2 Hz, 1 H), 5.41 (dd, J=9.2, 9.2 Hz, 1 H), 6.23 (br s, 1 H), 7.04-7.12 (m, 1 H), 7.18-7.31 (m, 5 H), 7.34-7.40 (m, 2 H), 8.23 (d, J=8.8 Hz, 1 H), 8.99 (s, 1 H); Anal. calcd. for C₂₂H₂₆ClN₅O: C, 64.15; H, 6.36; N, 17.00. Found: C, 63.98; H, 6.35; N, 16.96.

### Example 18

### N-[1-({(cyanoimino)[(2-methylphenyl)amino]methyl}amino)-2,2-dimethylpropyl]-4-phenylbutanamide

### Example 18A

### N-[1-(1H-1,2,3-benzotriazol-1-yl)-2,2-dimethylpropyl]-4-phenylbutanamide

1H-1,2,3-Benzotriazole, trimethylacetaldehyde and 4-phenylbutanamide were processed as described in example 1B to provide the title compound. MS (ESI⁺) m/z 351 (M+H)⁺.

### Example 18B

### N-[1-({(cyanoimino)[(2-methylphenyl)amino]methyl}amino)-2,2-dimethylpropyl]-4-phenylbutanamide

N″-Cyano-N-(2-methylphenyl)guanidine and example 18A were processed as described in example 1D to provide the title compound. mp 153-154° C.; MS (ESI⁺) m/z 406 (M+H)⁺; ¹H NMR (300 MHz, DMSO-d₆) δ 0.89 (s, 9 H), 1.74-1.87 (m, 2 H), 2.12-2.22 (m, 5 H), 2.56 (t, J=8.1 Hz, 2 H), 5.43 (dd, J=9.0, 9.0 Hz, 1 H), 6.21 (br s, 1 H), 7.08-7.33 (m, 9 H), 8.03 (d, J=7.5 Hz, 1 H), 9.09 (m, 1 H); Anal. calcd. for C₂₄H₃₁N₅O: C, 71.08; H, 7.71; N, 17.27. Found: C, 70.91; H, 7.66; N, 17.56.

### Example 19

### N-[1-({(cyanoimino)[(2,5-difluorophenyl)amino]methyl}amino)-2,2-dimethylpropyl]-2-(3,4-dimethoxyphenyl)acetamide

N″-Cyano-N-(2,5-difluorophenyl)guanidine, (prepared as described in WO 01/09096), and example 11A were processed as described in example 1D to provide the title compound. mp 190-191° C.; MS (ESI⁺) m/z 460 (M+H)⁺; ¹H NMR (300 MHz, DMSO-d₆) δ 0.92 (s, 9 H), 3.40 (d, 1 H, J=13.9 Hz), 3.50 (d, 1 H, J=13.9 Hz), 3.71 (s, 6 H), 5.42 (dd, 1 H, J=8.7 Hz), 6.77 (dd, 1 H, J=2.0, 8.1 Hz), 6.86-6.88 (m, 2H), 7.06-7.14(m, 2H), 7.19-7.26 (m, 1H), 7.29-7.37 (m, 1H), 8.12 (d, 1 H, J=7.1 Hz), 9.39 (s, 1 H); Anal. calcd. for C₂₃H₂₇F₂N₅O₃: C, 60.12; H, 5.92; N, 15.24. Found: C: 60.10, H: 6.06, N: 15.40.

### Example 20

### N-(1-{[[(2-chlorophenyl)amino](cyanoimino)methyl]amino}-2,2-dimethylpropyl)-2-(3,4-dimethoxyphenyl)acetamide

N″-Cyano-N-(2-chlorophenyl)guanidine, (prepared as described in Tilley et al. Helv. Chim. Acta.Vol 63 (4) pages 841-859 (1980)), and example 11A were processed as described in example 1D to provide the title compound. mp 135-136° C.; MS (ESI⁺) m/z 456 (M−H); ¹H NMR (300 MHz, DMSO-d₆) δ 0.91 (s, 9 H), 3.39 (d J=13.9 Hz, 1 H), 3.47 (d, J=13.9 Hz, 1 H), 3.72 (s, 6H), 5.42 (dd, J=8.7, 8.7 Hz, 1 H), 6.75-6.91 (m, 4 H), 7.24-7.40 (m, 3 H), 7.50-7.56 (m, 1 H), 8.17 (d, J=8.1 Hz, 1 H), 9.22 (s, 1 H); Anal. calcd. for C₂₃H₂₈ClN₅O₃: C, 60.32; H, 6.16; N, 15.29. Found: C, 60.09; H, 6.10; N, 15.57.

### Example 21

### N-[1-({(cyanoimino)[(2,5-dimethylphenyl)amino]methyl}amino)-2,2-dimethylpropyl]-2-(3,4-dimethoxyphenyl)acetamide

### Example 21A

### N″-cyano-N-(2,5-dimethylphenyl)guanidine

2,5-Dimethylaniline was processed as described in example 1C to provide the title compound. MS (ESI⁺) m/z 189 (M+H)⁺.

### Example 21B

### N-[1-({(cyanoimino)[(2,5-dimethylphenyl)amino]methyl}amino)-2,2-dimethylpropyl]-2-(3,4-dimethoxyphenyl)acetamide

Example 21A and example 11A were processed as described in example 1D to provide the title compound. mp 93-94° C.; MS (ESI⁺) m/z 452 (M+H)⁺; ¹H NMR (300 MHz, DMSO-d₆) δ 0.87 (s, 9 H), 2.08 (s, 3 H), 2.25 (s, 3 H), 3.38 (d, J=13.9 Hz, 1 H), 3.44 (d, J=13.9 Hz, 1 H), 3.69 (s, 3 H), 3.73 (s, 3 H), 5.41 (dd, J=9.2, 9.2 Hz, 1 H), 6.24 (br s, 1 H), 6.76 (dd, J=8.5, 3.0 Hz, 1 H), 6.85-6.90 (m, 3 H), 7.01 (d, J=8.1 Hz, 1 H), 7.14 (d, J=7.8 Hz, 1 H), 8.15 (d, J=8.1 Hz, 1 H), 8.99 (s, 1 H); Anal. calcd. for C₂₅H₃₃N₅O₃: C, 66.50; H, 7.37; N, 15.51. Found: C, 66.25; H, 7.43; N, 15.48.

### Example 22

### N-[1-({(cyanoimino)[(2,4,6-trifluorophenyl)amino]methyl}amino)-2,2-dimethylpropyl]-2-(3,4-dimethoxyphenyl)acetamide

### Example 22A

### N″-cyano-N-(2,4,6-trifluorophenyl)guanidine

2,4,6-Trifluoroaniline was processed as described in example 1C to provide the title compound. MS (ESI⁺) m/z 215 (M+H)⁺.

### Example 22B

### N-[1-({(cyanoimino)[(2,4,6-trifluorophenyl)amino]methyl}amino)-2,2-dimethylpropyl]-2-(3,4-dimethoxyphenyl)acetamide

Example 22A and example 11A were processed as described in example 1D to provide the title compound. mp 170-171° C.; MS (ESI⁺) m/z 478 (M+H)⁺; ¹H NMR (300 MHz, DMSO-d₆) δ 0.91 (s, 9 H), 3.40 (d, J=13.9 Hz, 1 H), 3.49 (d, J=13.9 Hz, 1 H), 3.72 (s, 6 H), 5.43 (dd, J=9.2, 9.2 Hz, 2 H), 6.74-6.89 (m, 3 H), 7.11 (br s, 1 H), 7.32 (dd, J=8.8, 8.8 Hz, 2 H), 8.02 (br s, 1 H), 8.99 (m, 1 H); Anal. calcd. for C₂₃H₂₆F₃N₅O₃: C, 57.86; H, 5.49; N, 14.67. Found: C, 58.15; H, 5.68; N, 14.63.

### Example 23

### N-(1-{[[(5-chloro-2-fluorophenyl)amino](cyanoimino)methyl]amino}-2,2-dimethylpropyl)-2-(3,4-dimethoxyphenyl)acetamide

### Example 23A

### N-(5-chloro-2-fluorophenyl)-N″-cyanoguanidine

2-Fluoro-5-chloroaniline was processed as described in example 1C to provide the title compound. MS (ESI⁺) m/z 213 (M+H)⁺.

### Example 23B

### N-(1-{[[(5-chloro-2-fluorophenyl)amino](cyanoimino)methyl]amino}-2,2-dimethylpropyl)-2-(3,4-dimethoxyphenyl)acetamide

Example 23A and example 11A were processed as described in example 1D to provide the title compound. mp 187-188° C.; MS (ESI⁺) m/z 476 (M+H)⁺; ¹H NMR (300 MHz, DMSO-d₆) δ 0.92 (s, 9 H), 3.40 (d, J=14.2 Hz, 1 H), 3.50 (d, J=14.2 Hz, 1 H), 3.71 (s, 6 H), 5.41 (dd, J=9.2, 9.2 Hz, 1 H), 6.77 (dd, J=8.1, 2.0 Hz, 1 H), 6.85-6.90 (m, 2 H), 7.13 (d, J=8.8 Hz, 1 H), 7.29-45 (m, 3 H), 8.11 (d, J=6.1 Hz, 1 H), 9.38 (m, 1 H); Anal. calcd. for C₂₃H₂₇ClFN₅O₃: C, 58.04; H, 5.72; N, 14.71. Found: C, 58.00; H, 5.72; N, 14.69.

### Example 24

### N-[1-({(cyanoimino)[(2-methoxy-3-pyridinyl)amino]methyl}amino)-2,2-dimethylpropyl]-2-(3,4-dimethoxyphenyl)acetamide

N″-Cyano-N-(2-methoxy-3-pyridinyl)guanidine, (prepared as described in U.S. 2002028836), and example 11A were processed as described in example 1D to provide the title compound. mp 146-147° C.; MS (ESI⁺) m/z 455 (M+H)⁺; ¹H NMR (300 MHz, DMSO-d₆) δ 0.89 (s, 9 H), 3.37 (d, J=13.9 Hz, 1 H), 3.46 (d, J=13.9 Hz, 2 H), 3.71 (s, 6 H), 3.87 (m, 3 H), 5.46 (dd, J=9.0, 9.0 Hz, 1 H), 6.73-6.89 (m, 4 H), 6.96-7.03 (m, 1 H), 7.59 (dd, J=7.5, 1.7 Hz, 1 H), 7.96-8.05 (m, 2 H), 8.91 (s, 1 H); Anal. calcd. for C₂₃H₃₀N₆O₄: C, 60.78; H, 6.53; N, 18.49. Found: C, 60.54; H, 6.56; N, 18.25.

### Example 25

### N-(1-{[[(2-chloro-3-pyridinyl)amino](cyanoimino)methyl]amino}-2,2-dimethylpropyl)-2-(3,4-dimethoxyphenyl)acetamide

N-(2-Chloro-3-pyridinyl)-N″-cyanoguanidine, (prepared as described in U.S. 2002028836), and example 11A were processed as described in example 1D to provide the title compound. MS (ESI⁺) m/z 459 (M+H)⁺; ¹H NMR (300 MHz, DMSO-d₆) δ 0.93 (s, 9 H), 3.40 (d, J=13.9 Hz, 1 H), 3.50 (d, J=13.6 Hz, 1 H), 3.73 (s, 6 H), 5.43 (dd, J=8.0, 8.0 Hz, 1 H), 6.78 (dd, J=8.3, 1.9 Hz, 1 H), 6.85-6.91 (m, 2 H), 7.16 (br s, 1 H), 7.39-7.46 (m, 1 H), 7.73 (d, J=7.1 Hz, 1 H), 8.16 (br s, 1 H), 8.26 (s, 1 H), 9.32 (s, 1 H); Anal. calcd. for C₂₂H₂₇ClN₆O₃0.1CH₂Cl₂: C, 57.27; H, 5.86; N, 18.22. Found: C, 57.41; H, 5.71; N, 17.88.

### Example 26

### 2-(4-chlorophenyl)-N-(1-{[[(2-chloro-3-pyridinyl)amino](cyanoimino)methyl]amino}-2,2-dimethylpropyl)acetamide

N-(2-Chloro-3-pyridinyl)-N″-cyanoguanidine and example 2A were processed as described in example 1D to provide the title compound. mp 174-175° C.; MS (ESI⁺) m/z 434 (M+H)⁺; ¹H NMR (300 MHz, DMSO-d₆) δ 0.93 (s, 9 H), 3.50 (d, J=14.2 Hz, 1 H), 3.58 (d, J=14.2 Hz, 1 H), 5.42 (dd, J=8.8, 8.8 Hz, 1 H), 7.17 (d, J=8.5 Hz, 1 H), 7.28 (d, J=8.5 Hz, 2 H), 7.37 (d, J=8.5 Hz, 2 H), 7.44 (dd, J=7.8, 4.8 Hz, 1 H), 7.75 (dd, J=8.0, 1.5 Hz, 1 H), 8.25 (m, 1 H), 8.28 (dd, J=4.6, 1.5 Hz, 1 H), 9.25 (s, 1 H); Anal. calcd. for C₂₀H₂₂Cl₂N₆O 0.04CH₂Cl₂: C, 55.11; H, 5.10; N, 19.24. Found: C, 54.75; H, 4.79; N, 19.13.

### Example 27

### N-[1-({(cyanoimino)[(2,6-dimethoxy-3-pyridinyl)amino]methyl}amino)-2,2-dimethylpropyl]-2-(3,4-dimethoxyphenyl)acetamide

### Example 27A

### N″-cyano-N-(2,6-dimethoxy-3-pyridinyl)guanidine

2,6-Dimethoxy-3-pyridinylamine was processed as described in example 1C to provide the title compound. MS (ESI⁺) m/z 222 (M+H)⁺.

### Example 27B

### N-[1-({(cyanoimino)[(2,6-dimethoxy-3-pyridinyl)amino]methyl}amino)-2,2-dimethylpropyl]-2-(3,4-dimethoxyphenyl)acetamide

Example 27A and example 11A were processed as described in example 1D to provide the title compound. mp 162-163° C.; MS (ESI⁺) m/z 485 (M+H)⁺; ¹H NMR (300 MHz, DMSO-d₆) δ 0.83 (s, 9 H), 3.35 (d, J=4.4 Hz, 1 H), 3.39 (d, J=4.4 Hz, 1 H), 3.71 (s, 3 H), 3.72 (s, 3 H,) 3.86 (s, 3 H), 3.88 (s, 3 H), 5.48 (dd, J=8.8. 8.8 Hz, 1 H), 6.36-6.44 (m, 2 H), 6.75 (dd, J=8.1, 2.0 Hz, 1 H), 6.82-6.91 (m, 2 H), 7.47 (d, J=8.5 Hz, 1 H), 7.83 (d, J=9.8 Hz, 1 H), 8.66 (s, 1 H); Anal. calcd. for C₂₄H₃₂N₆O₅: C, 59.49; H, 6.66; N, 17.34. Found: C, 59.24; H, 6.61; N, 17.49.

### Exampe 28

### N-(1-{[(cyanoimino)(5-quinolinylamino)methyl]amino}-2,2-dimethylpropyl)-4-(2-pyridinyl)butanamide

### Example 28A

### N-[1-(1H-1,2,3-benzotriazol-1-yl)-2,2-dimethylpropyl]-4-(2-pyridinyl)butanamide

1H-1,2,3-Benzotriazole, trimethylacetaldehyde and 4-(2-pyridinyl)butanamide, (prepared as described in Proft; Steinke; Chem. Ber., 94:2267-2270 (1961)), were processed as described in example 1B to provide the title compound. MS (ESI⁺) m/z 352 (M+H)⁺.

### Example 28B

### N″-cyano-N-5-quinolinylguanidine

5-Aminoquinoline was processed as described in example 1C to provide the title compound. MS (ESI⁺) m/z 212 (M+H)⁺.

### Example 28C

### N-(1-{[(cyanoimino)(5-quinolinylamino)methyl]amino}-2,2-dimethylpropyl)-4-(2-pyridinyl)butanamide

Example 28A and example 28B were processed as described in example 1D to provide the title compound. MS (ESI⁺) m/z 444 (M+H)⁺; ¹H NMR (300 MHz, DMSO-d₆) δ 0.93 (s, 9 H), 1.84-1.98 (m, 2 H), 2.18 (t, J=7.8 Hz, 2 H), 2.71 (t, J=7.5 Hz, 2 H), 5.53 (dd, J=9.0, 9.0 Hz, 1 H), 6.60 (d, J=9.2 Hz, 1 H), 7.17-7.25 (m, 2 H), 7.46 (d, J=6.8 Hz, 1 H), 7.53-7.60 (m, 1 H), 7.65-7.81 (m, 2 H), 7.91-8.01 (m, 2 H), 8.31 (d, J=8.5 Hz, 1 H), 8.44-8.50 (m, 1 H), 8.91-8.96 (m, 1 H), 9.64 (s, 1 H).

### Example 29

### N-(1-{[(cyanoimino)(5-quinolinylamino)methyl]amino}-2,2-dimethylpropyl)-4-(2-thienyl)butanamide

Example 28B and example 7A were processed as described in example 1D to provide the title compound. MS (ESI+) m/z 449 (M+H)⁺; ¹H NMR (300 MHz, DMSO-d₆) δ 0.91 (s, 9 H), 1.79-1.90 (m, 2 H), 2.20 (t, J=7.1 Hz, 2 H), 2.79 (t, J=7.8 Hz, 2 H), 5.53 (dd, J=9.0, 9.0 Hz, 1 H), 6.58 (br s, 1 H), 6.81-6.85 (m, 1 H), 6.92-6.96 (m, 1 H), 7.30-7.34(m, 1 H), 7.43-7.48 (m, 1 H), 7.52-7.60 (m, 1 H), 7.76 (t, J=8.5 Hz, 1 H), 7.87-8.01 (m, 2 H), 8.31 (d, J=8.5 Hz, 1 H), 8.91-8.95 (m, 1 H), 9.62 (s, 1 H).

### Example 30

### N-(1-{[(cyanoimino)(5-quinolinylamino)methyl]amino}-2,2-dimethylpropyl)-2-(3,4-dimethoxyphenyl)acetamide

Example 28B and example 11A were processed as described in example 1D to provide the title compound. mp 128-129° C.; MS (ESI⁺) m/z 475 (M+H)⁺; ¹H NMR (300 MHz, DMSO-d₆) δ 0.90 (m, 9 H), 3.39 (d, J=14.2 Hz, 1 H), 3.46 (d, J=13.9 Hz, 1 H), 3.71 (d, J=7.8 Hz, 6 H), 5.52 (dd, J=8.8, 8.8 Hz, 1 H), 6.67 (br d, J=8.1 Hz, 1 H), 6.77 (dd, J=8.1, 1.7 Hz, 1 H), 6.88 (m, 2 H), 7.41 (dd, J=7.5, 1.0 Hz, 1 H), 7.55 (dd, J=8.5, 4.1 Hz, 1 H), 7.75 (m, 1 H), 7.97 (d, J=8.5 Hz, 1 H), 8.06 (br d, J=6.8 Hz, 1 H), 8.26 (d, J=7.8 Hz, 1 H), 8.94 (dd, J=4.4, 1.7 Hz, 1 H), 9.59 (br s, 1 H); Anal. calcd. for C₂₆H₃₀N₆O₃0.09 CH₂Cl₂: C, 64.99; H, 6.31; N, 17.43. Found: C, 64.66; H, 5.93; N, 17.42.

### Example 31

### N-(1-{[(cyanoimino)(5-quinolinylamino)methyl]amino}-2,2-dimethylpropyl)-2-[4-(methylthio)phenyl]acetamide

Example 28B and example 8A were processed as described in example 1D to provide the title compound. mp 188-189° C.; MS (ESI⁺) m/z 461 (M+H)⁺; ¹H NMR (300 MHz, DMSO-d₆) δ 0.90 (s, 9 H), 2.45 (s, 3 H), 3.50 (d, J=12.6 Hz, 1 H), 3.44 (d, J=12.6 Hz, 1 H), 5.51 (dd, J=8.8, 8.8 Hz, 1 H), 6.67 (s, 1 H), 7.21 (s, 4 H), 7.40 (d, J=7.5 Hz, 1 H), 7.55 (dd, J=8.5, 4.07 Hz, 1 H), 7.74 (t, J=7.5 Hz, 1 H), 7.97 (d, J=8.5 Hz, 1 H), 8.12 (s, 1 H), 8.26 (d, J=8.5 Hz, 1 H), 8.94 (dd, J=4.1, 1.7 Hz, 1 H), 9.6 (s, 1 H); Anal. calcd. for C₂₅H₂₈N₆OS: C, 65.19; H, 6.13; N, 18.25. Found: C, 65.18; H, 6.07; N, 18.05.

### Example 32

### 2-(4-chlorophenyl)-N-(1-{[(cyanoimino)(5-quinolinylamino)methyl]amino}-2,2-dimethylpropyl)acetamide

Example 28B and example 2A were processed as described in example 1D to provide the title compound. mp 195-196° C.; MS (ESI⁺) m/z 449 (M+H)⁺; ¹H NMR (300 MHz, DMSO-d₆) δ 0.90 (s, 9 H), 3.48 (d, J=14.2 Hz, 1 H), 3.55 (d, J=14.6 Hz, 1 H), 5.50 (dd, J=8.8, 8.8 Hz, 1 H), 6.66 (d, J=8.1 Hz, 1 H), 7.28 (d, J=8.8 Hz, 2 H), 7.37 (d, J=8.8 Hz, 2 H), 7.40 (d, J=7.1 Hz, 1 H), 7.55 (dd, J=8.5, 4.1 Hz, 1 H), 7.75 (dd, J=8.5, 7.8 Hz, 1 H), 7.97 (d, J=8.8 Hz, 1 H), 8.15 (d, J=8.5 Hz, 1 H), 8.26 (d, J=8.5 Hz, 1 H), 8.94 (dd, J=4.1, 1.7 Hz, 1 H), 9.55 (s, 1 H); Anal. calcd. for C₂₄H₂₅ClN₆O: C, 64.21; H, 5.61; N, 18.72. Found: C, 63.97; H, 5.57; N, 18.52.

### Example 33

### N-(1-{[(cyanoimino)(5-quinolinylamino)methyl]amino}-2,2-dimethylpropyl)-4-phenylbutanamide

Example 28B and example 18A were processed as described in example 1D to provide the title compound. MS (ESI⁺) m/z 443 (M+H)⁺; ¹H NMR (300 MHz, DMSO-d₆) δ 0.91 (s, 9 H), 1.80 (m, 2 H), 2.16 (t, J=7.3 Hz, 2 H), 2.58 (m, 2 H), 5.53 (dd, J=8.8, 8.8 Hz, 2 H), 6.56 (d, J=8.8 Hz, 1 H), 7.17 (m, 3 H), 7.28 (m, 2 H), 7.45 (dd, J=7.1, 1.0 Hz, 1 H), 7.54 (dd, J=8.5, 4.1 Hz, 1 H), 7.75 (dd, J=8.3, 7.6 Hz, 1 H), 7.91 (s, 1 H), 7.96 (d, J=8.5 Hz, 1 H), 8.30 (d, J=8.1 Hz, 1 H), 8.92 (dd, J=4.1, 1.4 Hz, 1 H), 9.63 (s, 1 H).

### Example 34

### N-(1-{[(cyanoimino)(5-quinolinylamino)methyl]amino}-2,2-dimethylpropyl)-2-[4-(dimethylamino)phenyl]acetamide

### Example 34A

### N-[1-(1H-1,2,3-benzotriazol-1-yl)-2,2-dimethylpropyl]-2-[4-(dimethylamino)phenyl]acetamide

1H-1,2,3-Benzotriazole, trimethylacetaldehyde and 2-[4-(dimethylamino)phenyl]acetamide were processed as described in example 1B to provide the title compound. MS (ESI⁺) m/z 366 (M+H)⁺.

### Example 34B

### N-(1-{[(cyanoimino)(5-quinolinylamino)methyl]amino}-2,2-dimethylpropyl)-2-[4-(dimethylamino)phenyl]acetamide

Example 28B and example 34A were processed as described in example 1D to provide the title compound. MS (ESI⁺) m/z 458 (M+H)⁺; ¹H NMR (300 MHz, DMSO-d₆) δ 0.89 (s, 9 H), 2.85 (s, 6 H), 3.34 (d, J=13.9 Hz, 1 H), 3.39 (d, J=13.9 Hz, 1 H), 5.51 (dd, J=9.0, 9.0 Hz, 1 H), 6.66 (d, J=8.8 Hz, 3 H), 7.07 (d, J=8.5 Hz, 2 H), 7.41 (d, J=7.5 Hz, 1 H), 7.55 (dd, J=8.5, 4.1 Hz, 1 H), 7.75 (t, J=8.0 Hz, 1 H), 7.96 (d, J=8.5 Hz, 1 H), 8.01 (s, 1 H), 8.26 (d, J=7.8 Hz, 1 H), 8.94 (dd, J=4.1, 1.4 Hz, 1 H), 9.60 (s, 1 H).

### Example 35

### N-(1-{[(cyanoimino)(5-quinolinylamino)methyl]amino}-2,2-dimethylpropyl)-2-(1-naphthyl)acetamide

### Example 35A

### N-[1-(1H-1,2,3-benzotriazol-1-yl)-2,2-dimethylpropyl]-2-(1-naphthyl)acetamide

1H-1,2,3-Benzotriazole, trimethylacetaldehyde and 2-(1-naphthyl)acetamide were processed as described in example 1B to provide the title compound. MS (ESI⁺) m/z 373 (M+H)⁺.

### Example 35B

### N-(1-{[(cyanoimino)(5-quinolinylamino)methyl]amino}-2,2-dimethylpropyl)-2-(1-naphthyl)acetamide

Example 28B and example 35A were processed as described in example 1D to provide the title compound. MS (ESI⁺) m/z 465 (M+H)⁺; ¹H NMR (300 MHz, DMSO-d₆) δ 0.91 (s, 9 H), 3.97 (d, J=15.3 Hz, 1 H), 4.04 (d, J=15.3 Hz, 1 H), 5.54 (dd, J=9.0, 9.0 Hz, 1 H), 6.74 (d, J=9.5 Hz, 1 H), 7.37 (d, J=7.5 Hz, 1 H), 7.45 (m, 3 H), 7.51 (m, 2 H), 7.73 (t, J=7.8 Hz, 1 H), 7.84 (dd, J=5.9, 3.6 Hz, 1 H), 7.95 (m, 1 H), 8.06 (m, 1 H), 8.19 (d, J=8.8 Hz, 1 H), 8.26 (m, 1 H), 8.91 (dd, J=4.2, 1.2 Hz, 1 H), 9.55 (s, 1 H).

### Example 36

### N-(1-{[(cyanoimino)(5-quinolinylamino)methyl]amino}-2,2-dimethylpropyl)-2-(2-naphthyl)acetamide

### Example 36A

### N-[1-(1H-1,2,3-benzotriazol-1-yl)-2,2-dimethylpropyl]-2-(2-naphthyl)acetamide

1H-1,2,3-Benzotriazole, trimethylacetaldehyde and 2-(2-naphthyl)acetamide were processed as described in example 1B to provide the title compound. MS (ESI⁺) m/z 373 (M+H)⁺.

### Example 36B

### N-(1-{[(cyanoimino)(5-quinolinylamino)methyl]amino}-2,2-dimethylpropyl)-2-(2-naphthyl)acetamide

Example 28B and example 36A were processed as described in example 1D to provide the title compound. mp 200-201° C.; MS (ESI⁺) m/z 465 (M+H)⁺; ¹H NMR (300 MHz, DMSO-d₆) δ 0.91 (s, 9 H), 3.69 (s, 2 H), 5.55 (dd, J=8.8, 8.8 Hz, 1 H), 6.69 (d, J=8.1 Hz, 1 H), 7.39 (m, 1 H), 7.48 (m, 5 H), 7.70 (dd, J=8.5, 7.5 Hz, 1 H), 7.77 (s, 1 H), 7.86 (m, 3 H), 7.93 (m, 1 H), 8.18 (m, 1 H), 8.24 (d, J=8.1 Hz, 1 H), 8.90 (dd, J=4.2, 1.5 Hz, 2 H), 9.57 (br s, 1 H).

### Example 37

### N-(1-{[(cyanoimino)(5-quinolinylamino)methyl]amino}-2,2-dimethylpropyl)-2-[4-(ethylthio)phenyl]acetamide

### Example 37A

### 2-[4-(ethylthio)phenyl]acetamide

[4-(Ethylthio)phenyl]acetic acid 773 g, (prepared as described in Chem.Abstr. Vol. 63, page 14 (1965)), SOCl₂, and liquid NH₃ were processed as described in example 1A to provide the title compound. MS (ESI⁺) m/z 213 (M+NH₃)⁺.

### Example 37B

### N-[1-(1H-1,2,3-benzotriazol-1-yl)-2,2-dimethylpropyl]-2-[4-(ethylthio)phenyl]acetamide

1H-1,2,3-Benzotriazole, trimethylacetaldehyde and example 37A were processed as described in example 1B to provide the title compound. MS (ESI⁺) m/z 383 (M+H)⁺.

### Example 37C

### N-(1-{[(cyanoimino)(5-quinolinylamino)methyl]amino}-2,2-dimethylpropyl)-2-[4-(ethylthio)phenyl]acetamide

Example 28B and example 37B were processed as described in example 1D to provide the title compound. mp 189-190° C.; MS (ESI⁺) m/z 475 (M+H)⁺; ¹H NMR (300 MHz, DMSO-d₆) δ 0.90 (s, 9 H), 1.21 (t, J=7.1 Hz, 3 H), 2.89-3.00 (m, 2 H), 3.45 (d, J=14.6 Hz, 1 H), 3.51 (d, J=14.6 Hz, 1 H), 5.51 (dd, J=8.8, 8.8 Hz, 1 H), 6.66 (d, J=9.2 Hz, 1 H), 7.17-7.29 (m, 4 H), 7.41 (dd, J=0.7, 7.5 Hz, 1 H), 7.52-7.58 (m, 1 H), 7.75 (dd, J=7.5, 8.5 Hz, 1 H), 7.97 (d, J=8.5 Hz, 1 H), 8.13 (br s, 1 H), 8.26 (d, J=8.5 Hz, 1 H), 8.94 (dd, J=1.7, 4.1 Hz, 1 H); Anal. calcd. for C₂₆H₃₀N₆OS: C, 65.80; H, 6.37; N, 17.71. Found: C, 66.17; H, 6.22; N, 17.90.

### Example 38

### N-(1-{[(cyanoimino)(5-quinolinylamino)methyl]amino}-2,2-dimethylpropyl)-2-(2,3-dihydro-1,4-benzodioxin-6-yl)acetamide

### Example 38A

### 2-(2,3-dihydro-1,4-benzodioxin-6-yl)acetamide

2,3-Dihydro-1,4-benzodioxin-6-ylacetic acid (Vazquez et al., Farmaco. Vol. 51 page 3 (1996)), SOCl₂, and liquid NH₃ were processed as described in example 1A to provide the title compound. MS (ESI⁺) m/z 211 (M+NH₃)⁺.

### Example 38B

### N-[1-(1H-1,2,3-benzotriazol-1-yl)-2,2-dimethylpropyl]-2-(2,3-dihydro-1,4-benzodioxin-6-yl)acetamide

1H-1,2,3-Benzotriazole, trimethylacetaldehyde and example 38A were processed as described in example 1B to provide the title compound. MS (ESI⁺) m/z 381 (M+H)⁺.

### Example 38C

### N-(1-{[(cyanoimino)(5-quinolinylamino)methyl]amino}-2,2-dimethylpropyl)-2-(2,3-dihydro-1,4-benzodioxin-6-yl)acetamide

Example 28B and example 38B were processed as described in example 1D to provide the title compound. mp 191-192° C.; MS (ESI⁺) m/z 473 (M+H)⁺; ¹H NMR (300 MHz, DMSO-d₆) δ 0.91 (s, 9 H), 3.35-3.44 (m, 2 H), 4.21 (s, 4 H), 5.50 (dd, J=8.8, 8.8 Hz, 1 H), 6.64-6.73 (m, 2 H), 6.75-6.80 (m, 2 H), 7.42 (d, J=7.5 Hz, 1 H), 7.53-7.59 (m, 1 H), 7.75 (dd, J=7.5, 8.5 Hz, 1 H), 7.97 (d, J=8.5 Hz, 1 H), 8.07 (br s, 1 H), 8.27 (d, J=8.5 Hz, 1 H), 8.94 (dd, J=1.7, 4.1 Hz, 1 H), 9.59 (s, 1 H); Anal. calcd. for C₂₆H₂₈N₆O₃: C, 66.09; H, 5.97; N, 17.78. Found: C, 66.45; H, 5.67; N, 17.67.

### Example 39

### N-(1-{[(cyanoimino)(5-quinolinylamino)methyl]amino}-2,2-dimethylpropyl)-2-(7-quinolinyl)acetamide

### Example 39A

### 2-(7-quinolinyl)acetamide

Methyl 7-quinolinylacetate (2.128 g, 10.59 mmol), (prepared as described in Gielen et al., Tetrahedron Lett. Vol. 43(3) pages 419-422 (2002)), was dissolved in 7N NH₃ solution in MeOH (15 mL) and the solution was stirred at 60° C. in a sealed tube for 48 hr. The solvent and NH₃ excess were removed under reduced pressure to afford 1.026 g of the title compound. MS (ESI⁺) m/z 187 (M+H)⁺.

### Example 39B

### N-[1-(1H-1,2,3-benzotriazol-1-yl)-2,2-dimethylpropyl]-2-(7-quinolinyl)acetamide

1H-1,2,3-Benzotriazole, trimethylacetaldehyde and example 39A were processed as described in example 1B to provide the title compound. MS (ESI⁺) m/z 374 (M+H)⁺.

### Example 39C

### N-(1-{[(cyanoimino)(5-quinolinylamino)methyl]amino}-2,2-dimethylpropyl)-2-(7-quinolinyl)acetamide

Example 28B and example 39B were processed as described in example 1D to provide the title compound. mp 147-148° C.; MS (ESI⁺) m/z 466 (M+H)⁺; ¹H NMR (300 MHz, DMSO-d₆) δ 0.91 (s, 9 H), 3.73 (d, J=14.6 Hz, 1 H), 3.79 (d, J=14.6 Hz, 1 H), 5.55 (dd, J=8.8, 8.8 Hz, 1 H), 6.70 (d, J=9.2 Hz, 1 H), 7.40 (dd, J=1.0, 7.5, Hz, 1 H), 7.46-7.57 (m, 3 H), 7.67-7.75 (m, 1 H), 7.89-7.98 (m, 3 H), 8.21-8.30 (m, 2 H), 8.34 (dd, J=2.37, 9.15, Hz, 1 H), 8.87-8.93 (m, 2 H), 9.55 (s, 1 H); Anal. calcd. for C₂₇H₂₇N₇O0.12H₂O: C, 69.34; H, 5.87; N, 20.96. Found: C, 68.94; H, 5.76; N, 20.80.

### Example 40

### N-(1-{[(cyanoimino)(5-quinolinylamino)methyl]amino}-2,2-dimethylpropyl)-2-(6-quinolinyl)acetamide

### Example 40A

### N-[1-(1H-1,2,3-benzotriazol-1-yl)-2,2-dimethylpropyl]-2-(6-quinolinyl)acetamide

1H-1,2,3-Benzotriazole, trimethylacetaldehyde and 2-(6-quinolinyl)acetamide, (prepared as described in Tsatsaronis, Kehayoglou, J.Org.Chem. Vol. 35, page 438 (1970)), were processed as described in example 1B to provide the title compound. MS (ESI⁺) m/z 374 (M+H)⁺.

### Example 40B

### N-(1-{[(cyanoimino)(5-quinolinylamino)methyl]amino}-2,2-dimethylpropyl)-2-(6-quinolinyl)acetamide

Example 28B and example 40A were processed as described in example 1D to provide the title compound. mp 150-151° C.; MS (ESI⁺) m/z 466 (M+H)⁺; ¹H NMR (300 MHz, DMSO-d₆) δ 0.91 (s, 9 H), 3.74 (s, 2 H), 5.55 (dd, J=8.8, 8.8 Hz, 1 H), 6.68 (d, J=8.8 Hz, 1 H), 7.39 (dd, J=0.7, 8.1, Hz, 1 H), 7.44-7.55 (m, 2 H), 7.64-7.55 (m, 2 H), 7.80-7.86 (m, 1 H), 7.91-8.01 (m, 2 H), 8.17-8.33 (m, 3 H), 8.84-8.92 (m, 2 H), 9.56 (s, 1 H).

### Example 41

### N-(1-{[(cyanoimino)(5-quinolinylamino)methyl]amino}-2,2-dimethylpropyl)-2-(4-cyanophenyl)acetamide

### Example 41A

### N-[1-(1H-1,2,3-benzotriazol-1-yl)-2,2-dimethylpropyl]-2-(4-cyanophenyl)acetamide

1H-1,2,3-Benzotriazole, trimethylacetaldehyde and 2-(4-cyanophenyl)acetamide, (prepared as described in Mellinghoff, Chem.Ber. Vol. 2 page 3208 (1889)), were processed as described in example 1B to provide the title compound. MS (ESI⁺) m/z 348 (M+H)⁺.

### Example 41B

### N-(1-{[(cyanoimino)(5-quinolinylamino)methyl]amino}-2,2-dimethylpropyl)-2-(4-cyanophenyl)acetamide

Example 28B and example 41A were processed as described in example 1D to provide the title compound. mp 194.5-195.5° C.; MS (ESI⁺) m/z 440 (M+H)⁺; ¹H NMR (300 MHz, DMSO-d₆) δ 0.91 (s, 9 H), 3.59 (d, J=14.6 Hz, 1 H), 3.66 (d, J=14.6 Hz, 1 H), 5.50 (dd, J=8.8, 8.8 Hz, 1 H), 6.66 (d, J=9.2 Hz, 1 H), 7.38-7.49 (m, 3 H), 7.53-7.59 (m, 1 H), 7.71-7.81 (m, 3 H), 7.97 (d, J=8.5 Hz, 1 H), 8.17-8.31 (m, 2 H), 8.95 (dd, J=1.7, 5.8 Hz, 1 H), 9.53 (s, 1 H); Anal. calcd. for C₂₅H₂₅N₇O: C, 68.32; H, 5.73; N, 22.31. Found: C, 68.45; H, 5.77; N, 22.35.

### Example 42

### N-(1-{[(cyanoimino)(5-quinolinylamino)methyl]amino}-2,2-dimethylpropyl)-4-(3-pyridinyl)butanamide

### Example 42A

### N-[1-(1H-1,2,3-benzotriazol-1-yl)-2,2-dimethylpropyl]-4-(3-pyridinyl)butanamide

1H-1,2,3-Benzotriazole, trimethylacetaldehyde and 4-(3-pyridinyl)butanamide, (prepared as described in Mayer, Joachim M., Testa, Bernard, Helv. Chim. Acta, Vol. 65(6) pages 1868-1884 (1982)), were processed as described in example 1B to provide the title compound. MS (ESI⁺) m/z 352 (M+H)⁺.

### Example 42B

### N-(1-{[(cyanoimino)(5-quinolinylamino)methyl]amino}-2,2-dimethylpropyl)-4-(3-pyridinyl)butanamide

Example 28B and example 42A were processed as described in example 1D to provide the title compound. mp 181-182° C.; MS (ESI⁺) m/z 444 (M+H)⁺; ¹H NMR (300 MHz, DMSO-d₆) δ 0.93 (s, 9 H), 1.75-1.88 (m, 2 H), 2.17 (t, J=7.1 Hz, 2 H), 2.59 (t, J=7.5 Hz, 2 H), 5.52 (dd, J=8.8, 8.8 Hz, 1 H), 6.59 (br s, 1 H), 7.27-7.34 (m, 1 H), 7.46 (dd, J=1.0, 7.5, Hz, 1 H), 7.54-7.62 (m, 2 H), 7.76 (t, J=19.7 Hz, 1 H), 7.89-8/02 (m, 2 H), 8.32 (d, J=8.5 Hz, 1 H), 8.39-8.45 (m, 2 H), 8.94 (d, J=5.8 Hz, 1 H), 9.64 (s, 1 H); Anal. calcd. for C₂₅H₂₉N₇O: C, 67.70; H, 6.59; N, 22.11. Found: C, 67.67; H, 6.65; N, 22.19.

### Example 43

### 2-(4-chlorophenyl)-N-[1-({(cyanoimino)](1-methyl-1,2,3,4-tetrahydro-5-quinolinyl)amino]methyl}amino)-2,2-dimethylpropyl]acetamide

### Example 43A

### 1-methyl-1,2,3,4-tetrahydro-5-quinolinamine

The iodide salt of 5-amino-1-methylquinolinium (3 g, 9.5 mmol), (prepared as described in McCurdy et al., J. Am. Chem. Soc. Vol. 114(26) pages 10314-21 (1992)), in 250 ml of acetic acid was treated with PtO₂ (0.6 g) under hydrogen at 50 psi for 48 hr at room temperature. To the precipitated tan-colored product was added 100 ml of methanol to homogenize for filtration. Filtrate was concentrated, dissolved in methylene chloride, cooled, neutralized with 1M NaHCO₃ to pH 7, and finally extracted with methylene chloride. The combined organic layers were dried (Na₂SO₄), filtered, and the filtrate was concentrated to provide the title compound. MS (ESI⁺) m/z 163 (M+H)⁺.

### Example 43B

### N″-cyano-N-(1-methyl-1,2,3,4-tetrahydro-5-quinolinyl)guanidine

Example 43A was processed as described in example 1C to provide the title compound. MS (ESI⁺) m/z 230 (M+H)⁺.

### Example 43C

### 2-(4-chlorophenyl)-N-[1-({(cyanoimino)[(1-methyl-1,2,3,4-tetrahydro-5-quinolinyl)amino]methyl}amino)-2,2-dimethylpropyl]acetamide

Example 43B and 2A were processed as described in example 1D to provide the title compound. mp 114-117° C.; MS (ESI⁺) m/z 413 (M+H)⁺; ¹H NMR (300 MHz, DMSO-d₆) δ 0.82 (s, 9 H), 1.80 (m, 2 H), 2.49 (m, 2 H), 2.85 (s, 3 H), 3.16 (dd, J=6.3, 4.9 Hz, 2 H), 3.45 (s, 2 H) 5.41 (dd, J=9.0, 9.0 Hz, 1 H), 5.88 (br d, J=7.8 Hz, 1 H), 6.34 (dd, J=7.6, 0.9 Hz, 1 H), 6.55 (d, J=7.8 Hz, 1 H), 7.02 (t, J=8.0 Hz, 1 H), 7.26 (d, J=8.8 Hz, 1 H), 7.36 (d, J=8.8 Hz, 1 H), 8.18 (br d, J=8.5 Hz, 1 H), 8.82 (s, 1 H); Anal. calcd. for C₂₅H₃₁ClN₆O: C, 64.30; H, 6.69; N, 18.00. Found: C, 64.10; H, 6.87; N, 17.97

### Example 44

### 2-(4-chlorophenyl)-N-[1-({(cyanoimino)[(2-methyl-5-quinolinyl)amino]methyl}amino)-2,2-dimethylpropyl]acetamide

### Example 44A

### N″-cyano-N-(2-methyl-5-quinolinyl)guanidine

2-Methyl-5-quinolinamine was processed as described in example 1C to provide the title compound. MS (ESI⁺) m/z 226 (M+H)⁺.

### Example 44B

### 2-(4-chlorophenyl)-N-[1-({(cyanoimino)[(2-methyl-5-quinolinyl)amino]methyl}amino)-2,2-dimethylpropyl]acetamide

Example 44A and 2A were processed as described in example 1D to provide the title compound. mp 198-199° C.; MS (ESI⁺) m/z 464 (M+H)⁺; ¹H NMR (300 MHz, DMSO-d₆) δ 0.89 (s, 9 H), 2.68 (s, 3 H), 3.48 (d, J=14.6 Hz, 1 H), 3.54 (d, J=13.9 Hz, 1 H), 5.50 (dd, J=9.0, 9.0 Hz, 1 H), 6.64 (d, J=9.8 Hz, 1 H), 7.29 (d, J=8.1 Hz, 2 H), 7.33 (d, J=7.8 Hz, 1 H), 7.37 (d, J=8.5 Hz, 2 H), 7.44 (d, J=8.5 Hz, 1 H), 7.70 (dd, J=8.0, 7.5 Hz, 1 H), 7.86 (d, J=8.1 Hz, 1 H), 8.15 (d, J=8.8 Hz, 1 H), 8.19 (m, 1 H), 9.53 (s, 1 H).

### Example 45

### N-(1-{[(1,2-benzisothiazol-7-ylamino)(cyanoimino)methyl]amino}-2,2-dimethylpropyl)-2-(4-chlorophenyl)acetamide

### Example 45A

### N-1,2-benzisothiazol-7-yl-N″-cyanoguanidine

1,2-Benzisothiazol-7-amine, (prepared as described in Ricci et al., Ann.Chim., Vol. 53, pages 1860-1866 (1963)) was processed as described in example 1C to provide the title compound. MS (ESI⁺) m/z 218 (M+H)⁺.

### Example 45B

### N-(1-{[(1,2-benzisothiazol-7-ylamino)(cyanoimino)methyl]amino}-2,2-dimethylpropyl)-2-(4-chlorophenyl)acetamide

Example 45A and 2A were processed as described in example 1D to provide the title compound. mp 135-137° C.; MS (ESI⁺) m/z 455 (M)⁺; ¹H NMR (300 MHz, DMSO-d₆) δ 0.96 (s, 9 H), 3.52 (d, J=14.2 Hz, 1 H), 3.61 (d, J=14.2 Hz, 1 H), 5.45 (dd, J=8.8, 8.8 Hz, 1 H), 7.12 (d, J=9.5 Hz, 1 H), 7.28 (m, 3 H), 7.36 (d, J=8.8 Hz, 2 H), 7.52 (t, J=7.8 Hz, 1 H), 8.08 (d, J=8.1 Hz, 1 H), 8.34 (d, J=7.8 Hz, 1 H), 9.15 (s, 1 H), 9.88 (s, 1 H).

### Example 46

### N-(1-{[(cyanoimino)(5-isoquinolinylamino)methyl]amino}-2,2-dimethylpropyl)-2-[4-(methylthio)phenyl]acetamide

### Example 46A

### N″-cyano-N-5-isoquinolinylguanidine

5-Isoquinolinamine was processed as described in example 1C to provide the title compound. MS (ESI⁺) m/z 212 (M+H)⁺.

### Example 46B

### N-(1-{[(cyanoimino)(5-isoquinolinylamino)methyl]amino}-2,2-dimethylpropyl)-2-[4-(methylthio)phenyl]acetamide

Example 46A and 8A were processed as described in example 1D to provide the title compound. MS (ESI⁺) m/z 461 (M+H)⁺; ¹H NMR (300 MHz, DMSO-d₆) δ 0.92 (s, 9 H), 2.45 (s, 3 H), 3.46 (d, J=14.2 Hz, 1 H), 3.52 (d, J=13.9 Hz, 1 H), 5.50 (dd, J=8.8, 8.8 Hz, 1 H), 6.78 (d, J=8.5 Hz, 1 H), 7.22 (s, 3 H), 7.59 (d, J=8.5 Hz, 1 H), 7.66 (d, J=8.1 Hz, 1 H), 7.70 (d, J=6.1 Hz, 1 H), 8.06 (d, J=8.1 Hz, 1 H), 8.19 (d, J=8.8 Hz, 1 H), 8.19 (d, J=8.8 Hz, 1 H), 8.52 (d, J=6.1 Hz, 1 H), 9.36 (s, 1 H), 9.57 (s, 1 H).

### Example 47

### 2-(4-chlorophenyl)-N-(1-{[(cyanoimino)(5-isoquinolinylamino)methyl]amino}-2,2-dimethylpropyl)acetamide

Example 46A and 2A were processed as described in example 1D to provide the title compound. MS (ESI⁺) m/z 449 (M+H)⁺; ¹H NMR (300 MHz, DMSO-d₆) δ 0.92 (s, 9 H), 3.50 (d, J=14.2 Hz, 1 H), 3.57 (d, J=14.6 Hz, 1 H), 5.50 (dd, J=8.8, 8.8 Hz, 1 H), 6.78 (d, J=9.4 Hz, 1 H), 7.30 (d, J=8.5 Hz, 2 H), 7.37 (d, J=8.5 Hz, 2 H), 7.58 (d, J=7.1 Hz, 1 H), 7.67 (d, J=8.1 Hz, 1 H), 7.71 (dd, J=6.0, 1.0 Hz, 1 H), 8.06 (d, J=8.1 Hz, 1 H), 8.22 (d, J=8.1 Hz, 1 H), 8.52 (d, J=6.1 Hz, 1 H), 9.36 (s, 1 H), 9.55 (s, 1 H).

### Example 48

### N-(1-{[(cyanoimino)(5-isoquinolinylamino)methyl]amino}-2,2-dimethylpropyl)-2-[4-(dimethylamino)phenyl]acetamide

Examples 46A and 34A were processed as described in example 1D to provide the title compound. MS (ESI⁺) m/z 458 (M+H)⁺; ¹H NMR (300 MHz, DMSO-d₆) δ 0.91 (s, 9 H), 2.85 (s, 6 H), 3.37 (d, J=9.83 Hz, 1 H), 3.41 (d, J=9.83 Hz, 1 H), 5.48 (dd, J=9.2, 9.2 Hz, 1 H), 6.66 (d, J=8.82 Hz, 2 H), 6.78 (m, 1 H), 7.09 (d, J=8.82 Hz, 2 H), 7.58 (dd, J=7.46, 1.02 Hz, 1 H), 7.66 (d, J=7.80 Hz, 1 H), 7.71 (d, J=5.43 Hz, 1 H), 8.05 (d, J=6.78 Hz, 2 H), 8.52 (d, J=6.10 Hz, 1 H), 9.36 (s, 1 H), 9.62 (s, 1 H).

### Example 49

### N-(2,2-dichloro-1-{[(cyanoimino)(5-isoquinolinylamino)methyl]amino}propyl)-3,5-dimethoxybenzamide

Example 46A and N-[1-(1H-1,2,3-benzotriazol-1-yl)-2,2-dichloropropyl]-3,5-dimethoxybenzamide, (prepared as described in U.S. 2002028836), were processed as described in example 1D to provide the title compound. MS (ESI⁺) m/z 502 (M+H)⁺; ¹H NMR (300 MHz, DMSO-d₆) δ 2.11 (s, 3 H), 3.79 (s, 6 H), 6.53 (dd, J=8.6, 8.6 Hz, 1 H), 6.73 (m, 2 H), 6.92 (d, J=2.2 Hz, 2 H), 7.76 (m, 3 H), 8.16 (d, J=7.0 Hz, 1 H), 8.55 (d, J=5.9 Hz, 1 H), 8.64 (d, J=8.1 Hz, 1 H), 9.40 (s, 1 H), 10.04 (s, 1 H).

### Example 50

### N-[2,2-dichloro-1-({(cyanoimino)[(2-methoxy-3-pyridinyl)amino]methyl}amino)propyl]-3,5-dimethoxybenzamide

Example 24A and N-[1-(1H-1,2,3-benzotriazol-1-yl)-2,2-dichloropropyl]-3,5-dimethoxybenzamide were processed as described in example 1D to provide the title compound. mp 198-199° C.; MS (ESI⁺) m/z 482 (M+H)⁺; ¹H NMR (300 MHz, DMSO-d₆) δ 2.15 (s, 3 H), 3.80 (s, 6 H), 3.86 (s, 3 H), 6.48 (dd, J=8.7, 8.7 Hz, 1 H), 6.74 (t, J=2.2 Hz, 1 H), 6.79 (d, J=8.8 Hz, 1 H), 6.95 (d, J=2.4 Hz, 2 H), 7.07 (dd, J=7.6, 4.9 Hz, 1 H), 7.63 (dd, J=7.6, 1.5 Hz, 1 H), 8.14 (dd, J=4.9, 1.9 Hz, 1 H), 8.65 (d, J=8.1 Hz, 1 H), 9.53 (s, 1 H); Anal. calcd. for C₂₀H₂₂Cl₂N₆O₄: C, 49.91; H, 4.61; N, 17.46. Found: C, 49.96; H, 4.56; N, 17.43.

### Example 51

### 4-chloro-N-[2,2-dichloro-1-({(cyanoimino)[(2,6-dimethoxy-3-pyridinyl)amino]methyl}amino)propyl]benzamide

Example 26A and N-[1-(1H-1,2,3-benzotriazol-1-yl)-2,2-dichloropropyl]-4-chlorobenzamide, (prepared as described in WO 01/09096), were processed as described in example 1D to provide the title compound. mp 162-163° C.; MS (ESI⁺) m/z 486 (M+H)⁺; ¹H NMR (300 MHz, DMSO-d₆) δ 2.14 (s, 3 H), 3.85 (s, 3 H), 3.89 (s, 3 H), 6.45 (d, J=8.5 Hz, 1 H), 6.50 (dd, J=8.5, 8.5 Hz, 1 H), 6.59 (d, J=8.8 Hz, 1 H), 7.55 (d, J=8.1 Hz, 1 H), 7.62 (d, J=8.5 Hz, 2 H), 7.83 (d, J=8.8 Hz, 2 H), 8.70 (d, J=8.8 Hz, 1 H), 9.21 (s, 1 H); Anal. calcd. for C₁₉H₁₉Cl₃N₆O₃ 0.1C₆H₁₄: C, 47.62; H, 4.16; N, 17.00. Found: C, 47.68; H, 4.00; N, 17.04.

### Example 52

### 4-chloro-N-[2,2-dichloro-1-({(cyanoimino)[(2-methoxy-3-pyridinyl)amino]methyl}amino)propyl]benzamide

N″-Cyano-N-(2-methoxy-3-pyridinyl)guanidine and N-[1-(1H-1,2,3-benzotriazol-1-yl)-2,2-dichloropropyl]-4-chlorobenzamide were processed as described in example 1D to provide the title compound. mp 155-156° C.; MS (ESI⁺) m/z 457 (M+H)⁺; ¹H NMR (300 MHz, DMSO-d₆) δ 2.16 (s, 3 H), 3.86 (s, 3 H), 6.50 (dd, J=8.7 Hz, 1 H), 6.82 (d, J=8.8 Hz, 1 H), 7.07 (dd, J=7.5, 5.1 Hz, 1 H), 7.63 (m, 3 H), 7.84 (d, J=8.8 Hz, 2 H), 8.14 (dd, J=5.1, 1.7 Hz, 1 H), 8.77 (d, J=8.8 Hz, 1 H) 9.45 (s, 1 H); Anal. calcd. for C₁₈H₁₇Cl₃N₇O: C, 47.44; H, 3.76; N, 18.44. Found: C, 47.44; H, 3.93; N, 18.38.

### Example 53

### 4-chloro-N-(2,2-dichloro-1-{[[(2-chloro-3-pyridinyl)amino](cyanoimino)methyl]amino}propyl)benzamide

N-(2-Chloro-3-pyridinyl)-N″-cyanoguanidine and N-[1-(1H-1,2,3-benzotriazol-1-yl)-2,2-dichloropropyl]-4-chlorobenzamide were processed as described in example 1D to provide the title compound. mp 135-137° C.; MS (ESI⁺) m/z 461 (M+H)⁺; ¹H NMR (300 MHz, DMSO-d₆) δ 2.17 (s, 3 H), 6.50 (dd, J=8.7, 8.7 Hz, 1 H), 7.16 (d, J=8.5 Hz, 1 H), 7.52 (dd, J=7.8, 4.8 Hz, 1 H), 7.63 (d, J=8.8 Hz, 2 H), 7.86 (d, J=8.5 Hz, 2 H), 7.90 (m, 1 H), 8.39 (dd, J=4.8, 1.7 Hz, 1 H), 8.90 (d, J=8.5 Hz, 1 H), 9.86 (s, 1 H); Anal. calcd. for C₁₇H₁₄Cl₄N₆O: C, 44.37; H, 3.07; N, 18.26. Found: C, 44.44; H, 2.99; N, 18.19.

### Example 54

N-[2,2-dichloro-1-({(cyanoimino)[(2-methoxy-3-pyridinyl)amino]methyl}amino)propyl]-2-(3,4-dimethoxyphenyl)acetamide

### Example 54A

### N-[1-(1H-1,2,3-benzotriazol-1-yl)-2,2-dichloropropyl]-2-(3,4-dimethoxyphenyl)acetamide

1-(2,2-Dichloropropanoyl)-1H-1,2,3-benzotriazole and 2-(3,4-dimethoxyphenyl)acetamide, prepared as described in Kaufmann; Mueller; Chem. Ber., Vol. 51, page 123 (1918), were processed as described in example 1B to provide the title compound. MS (ESI⁺) m/z 424 (M+H)⁺.

### Example 54B

### N-[2,2-dichloro-1-({(cyanoimino)[(2-methoxy-3-pyridinyl)amino]methyl}amino)propyl]-2-(3,4-dimethoxyphenyl)acetamide

N″-Cyano-N-(2-methoxy-3-pyridinyl)guanidine and 54A were processed as described in example 1D to provide the title compound. mp 188-190° C.; MS (ESI⁺) m/z 496 (M+H)⁺; ¹H NMR (300 MHz, DMSO-d₆) δ 2.07 (s, 3 H), 3.43 (d, J=14.6 Hz, 1 H), 3.50 (d, J=14.9 Hz, 1 H), 3.71 (s, 3 H), 3.72 (s, 3 H), 3.89 (s, 3 H), 6.25 (dd, J=8.8, 8.8 Hz, 1 H), 6.77 (dd, J=8.1, 2.0 Hz, 1 H), 6.86 (m, 2 H), 7.04 (dd, J=7.5, 5.1 Hz, 2 H), 7.58 (dd, J=7.8, 1.4 Hz, 1 H), 8.09 (dd, J=4.9, 1.9 Hz, 1 H), 8.43 (d, J=9.2 Hz, 1 H), 9.26 (s, 1 H); Anal. calcd. for C₂₁H₂₄Cl₂N₆O₄: C, 50.92; H, 4.88; N, 16.97. Found: C, 51.01; H, 4.96; N, 17.13.

### Determination of Biological Activity

P2X₇ Mediated Pore Formation. Activation of the P2X₇ receptor induces nonspecific pore formation and eventually cell lysis (Verhoef et al., The Journal of Immunology, Vol. 170, pages 5728-5738, 2003). Accordingly, the inhibitory activity of the antagonists of the present invention was determined by their capacity to inhibit the agonist-induced pore formation using the fluorescent dye YO-PRO (MW=629) and Fluorescence Imaging Plate Reader (FLIPR, Molecular Devices, Sunnydale, Calif.) in THP-1 cells. Prior to YO-PRO dye addition, the cells were rinsed once in PBS without Mg²⁺ or Ca²⁺ ions, which have been shown to inhibit pore formation (Bianchi et al., Eur. J. Pharmacol. Vol 376, pages 127-138, 1999). The YO-PRO iodide dye (1 mM in DMSO) was diluted to a final concentration of 2 μM in PBS (w/o Mg²⁺ or Ca²⁺) and then placed on the cells immediately prior to the agonist addition. Since the THP-1 cells are a non-adherent cell line, the cells were washed in PBS and loaded with the dye in a conical tube prior to spinning the cells onto poly-lysine-coated black-walled 96-well plates, which were utilized to reduce light scattering. After the addition of the agonist BzATP (50 μM, the EC₇₀ value for agonist activation), the YO-PRO dye uptake was observed in the FLIPR apparatus equipped with an Argon laser (wavelength=480 nm) and a CCD camera. The intensity of the fluorescence was captured by the CCD camera every 15 seconds for the first 10 minutes of agonist exposure followed by every 20 seconds for an additional 50 minutes with the data being digitally transferred to an interfaced PC. The exposure setting of the camera was 0.25 sec with an f-stop setting of 2. For antagonist activity measurements, the percent maximal intensity was normalized to that induced by 50 μM BzATP plotted against each concentration of compound to calculate IC₅₀ values and account for plate-to-plate variability.

The potency of the compounds was inversely proportional to their IC₅₀ value. Representative compounds from the present invention had IC₅₀ values ranging from 0.01 to 0.29 nM.

2) P2X₇ Mediated IL-1β Release. Activation of P2X₇ receptors also induces secretion of IL-1β (Verhoef et al., above; Brough et al., Molecular and Cellular Neuroscience Vol. 19, pages 272-280, 2002). THP-1 cells were plated in 24-well plates at a density of 1×10⁶ cells/well/ml. On the day of the experiment, cells were differentiated with 25 ng/ml LPS and 10 ng/ml final concentration of γIFN for 3 hours at 37° C. In the presence of the differentiation media, the cells were incubated with the antagonists of the present invention for 30 minutes at 37° C. followed by a challenge with 1 mM BzATP for an additional 30 minutes at 37° C. Supernatants of the samples were collected after a 5 minutes centrifugation in microfuge tubes to pellet the cells and debris and to test for mature IL-1β released into the supernatant using either R & D Systems Human IL-1β ELISA assay or Endogen Human IL-1β ELISA, following the manufacturer's instructions. The concentration of the antagonists that inhibited 50% of the agonis-release of IL-1β was expressed as IC₅₀. Representative compounds of the present invention exhibited IC₅₀ values from 0.20 to 1.73 μM.

### Determination of Analgesic Activity

Adult male Sprague-Dawley rats (250-300 g), Charles River Laboratories, Portage, Mich. were used in this study. Animal handling and experimental protocols were approved by the Institutional Animal Care and Use Committee (IACUC) at Abbott Laboratories. For all surgical procedures, animals were maintained under halothane anesthesia (4% to induce, 2% to maintain), and the incision sites were sterilized using a 10% povidone-iodine solution prior to and after surgeries.

Spinal Nerve ligation. A model of spinal nerve ligation-induced neuropathic pain was produced using the procedure originally described by Kim and Chung (Kim and Chung, Pain, Vol. 50 pages 355-363, 1992). The left L5 and L6 spinal nerves of the rat were isolated adjacent to the vertebral column and tightly ligated with a 5-0 silk suture distal to the DRG, and care was taken to avoid injury of the L4 spinal nerve. Sham rats underwent the same procedure, but without nerve ligation. Dose-response curves as well as single dose responses were performed. All animals were allowed to recover for at least 1 week and not more than 3 weeks prior to assessment of mechanical allodynia. Representative compounds of the present invention exhibited ED₅₀ values between 35 and 159 μmol/kg, obtained from dose-response curves and intraperitoneal administration. ED₅₀ values after oral administration were between 2-10 times higher.

Sciatic nerve ligation. A model of chronic constriction injury-induced neuropathic pain was produced by following the method of Bennett and Xie (Bennet and Xie, Pain, Vol 33, pages 87-107, 1988). The right common sciatic nerve was isolated at mid-thigh level, and loosely ligated by 4 chromic gut (5-0) ties separated by an interval of 1 mm. Sham rats underwent the same procedure, but without sciatic nerve constriction. All animals were left to recover for at least 2 weeks and no more than 5 prior to testing of mechanical allodynia. Representative compounds exhibited ED₅₀ values of 242 and 227 mmol/kg, ip.

Chemotherapy induced neuropathic pain. A model of chemotherapy-induced neuropathic pain was produced by continuous intravenous (i.v.) vincristine infusion (Nozaki-Taguchi et al. Pain, Vol 93, pages 68-76, 2001). In this model, anesthetized rats underwent a surgical procedure consisting of jugular vein catheterization and subcutaneous implantation of a vincristine-primed mini-pump. Fourteen-day i.v. infusion of vincristine (30 μg/kg/day) resulted in systemic neuropathic pain of the animal. Sham rats underwent the same procedure, but with physiological saline infusion. Mechanical allodynia of the left hind paw was examined 7-21 days post implantation of mini-pumps. Mechanical allodynia was measured using calibrated von Frey filaments (Stoelting, Wood Dale, Ill.). Rats were placed into inverted individual plastic containers (20×12.5×20 cm) on top of a suspended wire mesh grid, and acclimated to the test chambers for 20 min. The von Frey filaments were presented perpendicularly to the plantar surface of the selected hind paw, and then held in this position for approximately 8 sec with enough force to cause a slight bend in the filament. Positive responses included an abrupt withdrawal of the hind paw from the stimulus, or flinching behavior immediately following removal of the stimulus. A 50% withdrawal threshold was determined using an up-down procedure (Dixon, Ann. Rev. Pharmacol. Toxicol., Vol. 20, pages 441-462, 1980). Prior to compound administration, animals demonstrating motor deficit or failure to exhibit subsequent mechanical allodynia were excluded from further studies.

Thirty minutes after intraperitoneal injection in appropriate vehicle, the effects of P2X7 receptor antagonists on mechanical allodynia observed after spinal nerve injury were evaluated.

1. A method of treating neuropathic pain comprising administering a therapeutically effective amount of a compound of formula I: 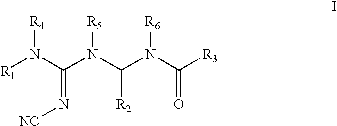
or a pharmaceutically acceptable salt, amide, ester or prodrug thereof, wherein
R₁ is selected from the group consisting of aryl, arylalkyl, heterocycle and heterocyclealkyl;
R₂ is selected from the group consisting of alkyl, and haloalkyl;
R₃ is selected from the group consisting of alkyl, aryl, arylalkyl, heterocycle and heterocyclealkyl; and
R₄, R₅, and R₆ are selected from the group consisting of hydrogen and alkyl.

2. The method according to claim 1 wherein
R₁ is aryl;
R₂ is alkyl; and
R₃ is arylalkyl.

3. The method according to claim 1 wherein
R₁ is aryl wherein the aryl is phenyl optionally substituted with 1, 2, or 3 substituents independently selected from the group consisting of alkyl and halogen;
R₂ is alkyl wherein the alkyl is tert-butyl; and
R₃ is arylalkyl wherein the aryl of the arylalkyl is selected from the group consisting of naphthyl and phenyl, wherein the phenyl is optionally substituted with 1 or 2 substituents indpendently selected from the group consisting of alkoxy, alkyl, alkylthio, cyano, ethylenedioxy, halogen, methylenedioxy, and R_{A}R_{B}N—.

4. The method according to claim 3 wherein the compound is selected from the group consisting of:
N-[1-({(cyanoimino)[(2-methylphenyl)amino]methyl}amino)-2,2-dimethylpropyl]-4-(4-methoxyphenyl)butanamide;
N-[1-({(cyanoimino)[(2-methylphenyl)amino]methyl}amino)-2,2-dimethylpropyl]-2-(3,4-dimethoxyphenyl)acetamide;
(−) N-[1-({(cyanoimino)[(2-methylphenyl)amino]methyl}amino)-2,2-dimethylpropyl]-2-(3,4-dimethoxyphenyl)acetamide;
(+) N-[1-({(cyanoimino)[(2-methylphenyl)amino]methyl}amino)-2,2-dimethylpropyl]-2-(3,4-dimethoxyphenyl)acetamide;
N-[1-({(cyanoimino)[(2-methylphenyl)amino]methyl}amino)-2,2-dimethylpropyl]-2-(4-methoxyphenyl)acetamide;
2-(1,3-benzodioxol-5-yl)-N-[1-({(cyanoimino)[(2-methylphenyl)amino]methyl}amino)-2,2-dimethylpropyl]acetamide;
N-[1-({(cyanoimino)[(2-methylphenyl)amino]methyl}amino)-2,2-dimethylpropyl]-2-[4-(methylthio)phenyl]acetamide;
2-(4-chlorophenyl)-N-[1-({(cyanoimino)[(2-methylphenyl)amino]methyl}amino)-2,2-dimethylpropyl]acetamide;
N-[1-({(cyanoimino)[(2-methylphenyl)amino]methyl}amino)-2,2-dimethylpropyl]-4-phenylbutanamide;
N-[1-({(cyanoimino)[(2,5-difluorophenyl)amino]methyl}amino)-2,2-dimethylpropyl]-2-(3,4-dimethoxyphenyl)acetamide;
N-(1-{[[(2-chlorophenyl)amino](cyanoimino)methyl]amino}-2,2-dimethylpropyl)-2-(3,4-dimethoxyphenyl)acetamide;
N-[1-({(cyanoimino)[(2,5-dimethylphenyl)amino]methyl}amino)-2,2-dimethylpropyl]-2-(3,4-dimethoxyphenyl)acetamide;
N-[1-({(cyanoimino)[(2,4,6-trifluorophenyl)amino]methyl}amino)-2,2-dimethylpropyl]-2-(3,4-dimethoxyphenyl)acetamide; and
N-(1-{[[(5-chloro-2-fluorophenyl)amino](cyanoimino)methyl]amino}-2,2-dimethylpropyl)-2-(3,4-dimethoxyphenyl)acetamide.

5. The method according to claim 1 wherein
R₁ is heterocycle;
R₂ is alkyl; and
R₃ is arylalkyl.

6. The method according to claim 1 wherein
R₁ is heterocycle wherein the heterocycle is selected from the group consisting of benzoisothiazolyl, isoquinolinyl, pyridinyl, quinolinyl, and tetrahydroquinolinyl, wherein the heterocycle is optionally substituted with 1 or 2 substituents independently selected from the group consisting of alkyl, alkoxy, and halogen;
R₂ is alkyl wherein the alkyl is tert-butyl; and
R₃ is arylalkyl wherein the aryl of the arylalkyl is selected from the group consisting of naphthyl and phenyl, wherein the phenyl is optionally substituted with 1 or 2 substituents independently selected from the group consisting of alkoxy, alkyl, alkylthio, cyano, ethylenedioxy, halogen, methylenedioxy, and R_{A}R_{B}N—.

7. The method according to claim 6 wherein the compound is selected from the group consisting of:
N-[1-({(cyanoimino)[(2-methyl-3-pyridinyl)amino]methyl}amino)-2,2-dimethylpropyl]-2-(3,4-difluorophenyl)acetamide;
2-(4-chlorophenyl)-N-[1-({(cyanoimino)[(2-methyl-3-pyridinyl)amino]methyl}amino)-2,2-dimethylpropyl]acetamide;
N-[1-({(cyanoimino)[(2-methyl-3-pyridinyl)amino]methyl}amino)-2,2-dimethylpropyl]-4-(4-methoxyphenyl)butanamide;
N-[1-({(cyanoimino)[(2-methyl-3-pyridinyl)amino]methyl}amino)-2,2-dimethylpropyl]-2-(3-fluoro-4-methylphenyl)acetamide;
N-[1-({(cyanoimino)[(2-methyl-3-pyridinyl)amino]methyl}amino)-2,2-dimethylpropyl]-5-phenylpentanamide;
N-[1-({(cyanoimino)[(2-methyl-3-pyridinyl)amino]methyl}amino)-2,2-dimethylpropyl]-2-(4-fluorophenyl)acetamide;
N-[1-({(cyanoimino)[(2-methyl-3-pyridinyl)amino]methyl}amino)-2,2-dimethylpropyl]-2-[4-(methylthio)phenyl]acetamide;
2-(4-chlorophenyl)-N-[1-({(cyanoimino)[(2-ethyl-3-pyridinyl)amino]methyl}amino)-2,2-dimethylpropyl]acetamide;
N-[1-({(cyanoimino)[(2-methoxy-3-pyridinyl)amino]methyl}amino)-2,2-dimethylpropyl]-2-(3,4-dimethoxyphenyl)acetamide;
N-(1-{[[(2-chloro-3-pyridinyl)amino](cyanoimino)methyl]amino}-2,2-dimethylpropyl)-2-(3,4-dimethoxyphenyl)acetamide;
2-(4-chlorophenyl)-N-(1-{[[(2-chloro-3-pyridinyl)amino](cyanoimino)methyl]amino}-2,2-dimethylpropyl)acetamide;
N-[1-({(cyanoimino)[(2,6-dimethoxy-3-pyridinyl)amino]methyl}amino)-2,2-dimethylpropyl]-2-(3,4-dimethoxyphenyl)acetamide;
N-(1-{[(cyanoimino)(5-quinolinylamino)methyl]amino}-2,2-dimethylpropyl)-2-(3,4-dimethoxyphenyl)acetamide;
N-(1-{[(cyanoimino)(5-quinolinylamino)methyl]amino}-2,2-dimethylpropyl)-2-[4-(methylthio)phenyl]acetamide;
2-(4-chlorophenyl)-N-(1-{[(cyanoimino)(5-quinolinylamino)methyl]amino}-2,2-dimethylpropyl)acetamide;
N-(1-{[(cyanoimino)(5-quinolinylamino)methyl]amino}-2,2-dimethylpropyl)-4-phenylbutanamide;
N-(1-{[(cyanoimino)(5-quinolinylamino)methyl]amino}-2,2-dimethylpropyl)-2-[4-(dimethylamino)phenyl]acetamide;
N-(1-{[(cyanoimino)(5-quinolinylamino)methyl]amino}-2,2-dimethylpropyl)-2-(1-naphthyl)acetamide;
N-(1-{[(cyanoimino)(5-quinolinylamino)methyl]amino}-2,2-dimethylpropyl)-2-(2-naphthyl)acetamide;
N-(1-{[(cyanoimino)(5-quinolinylamino)methyl]amino }-2,2-dimethylpropyl)-2-[4-(ethylthio)phenyl]acetamide;
N-(1-{[(cyanoimino)(5-quinolinylamino)methyl]amino}-2,2-dimethylpropyl)-2-(2,3-dihydro-1,4-benzodioxin-6-yl)acetamide;
N-(1-{[(cyanoimino)(5-quinolinylamino)methyl]amino}-2,2-dimethylpropyl)-2-(4-cyanophenyl)acetamide;
2-(4-chlorophenyl)-N-[1-({(cyanoimino)[(1-methyl-1,2,3,4-tetrahydro-5-quinolinyl)amino]methyl}amino)-2,2-dimethylpropyl]acetamide;
2-(4-chlorophenyl)-N-[1-({(cyanoimino)[(2-methyl-5-quinolinyl)amino]methyl}amino)-2,2-dimethylpropyl]acetamide;
N-(1-{[(1,2-benzisothiazol-7-ylamino)(cyanoimino)methyl]amino}-2,2-dimethylpropyl)-2-(4-chlorophenyl)acetamide;
N-(1-{[(cyanoimino)(5-isoquinolinylamino)methyl]amino}-2,2-dimethylpropyl)-2-[4-(methylthio)phenyl]acetamide;
2-(4-chlorophenyl)-N-(1-{[(cyanoimino)(5-isoquinolinylamino)methyl]amino}-2,2-dimethylpropyl)acetamide; and
N-(1-{[(cyanoimino)(5-isoquinolinylamino)methyl]amino}-2,2-dimethylpropyl)-2-[4-(dimethylamino)phenyl]acetamide.

8. The method according to claim 1 wherein
R₁ is heterocycle;
R₂ is alkyl; and
R₃ is heterocyclealkyl.

9. The method according to claim 1 wherein the
R₁ is heterocycle wherein the heterocycle is selected from the group consisting of pyridinyl and quinolinyl, wherein the heterocycle is optionally substituted with 1 alkyl substituent;
R₂ is alkyl wherein the alkyl is tert-butyl; and
R₃ is heterocyclealkyl wherein the heterocycle of the heterocyclalkyl is selected from the group consisting of pyridinyl, quinolinyl, and thienyl.

10. The method according to claim 9 wherein the compound is selected from the group consisting of:
N-[1-({(cyanoimino)[(2-methyl-3-pyridinyl)amino]methyl}amino)-2,2-dimethylpropyl]-4-(2-thienyl)butanamide;
N-(1-{[(cyanoimino)(5-quinolinylamino)methyl]amino}-2,2-dimethylpropyl)-4-(2-pyridinyl)butanamide;
N-(1-{[(cyanoimino)(5-quinolinylamino)methyl]amino}-2,2-dimethylpropyl)-4-(2-thienyl)butanamide;
N-(1-{[(cyanoimino)(5-quinolinylamino)methyl]amino}-2,2-dimethylpropyl)-2-(7-quinolinyl)acetamide;
N-(1-{[(cyanoimino)(5-quinolinylamino)methyl]amino}-2,2-dimethylpropyl)-2-(6-quinolinyl)acetamide; and
N-(1-{[(cyanoimino)(5-quinolinylamino)methyl]amino}-2,2-dimethylpropyl)-4-(3-pyridinyl)butanamide.

11. The method according to claim 1, wherein
R₁ is heterocycle;
R₂ is haloalkyl; and
R₃ is aryl.

12. The method according to claim 1 wherein
R₁ is heterocycle wherein the heterocycle is selected from the group consisting of isoquinolinyl and pyridinyl, wherein the heterocycle is optionally substituted with 1 or 2 substituents independently selected from the group consisting of alkoxy and halogen;
R₂ is haloalkyl wherein the haloalkyl is 1,1-dichloroethyl; and
R₃ is aryl wherein the aryl is selected from the group consisting of naphthyl and phenyl, wherein the phenyl is optionally substituted with 1 or 2 substituents independently selected from the group consisting of alkoxy, alkyl, alkylthio, cyano, ethylenedioxy, halogen, methylenedioxy, and R_{A}R_{B}N—.

13. The method according to claim 12 wherein the compound is selected from the group consisting of:
N-(2,2-dichloro-1-{[(cyanoimino)(5-isoquinolinylamino)methyl]amino}propyl)-3,5-dimethoxybenzamide;
N-[2,2-dichloro-1-({(cyanoimino)[(2-methoxy-3-pyridinyl)amino]methyl}amino)propyl]-3,5-dimethoxybenzamide;
4-chloro-N-[2,2-dichloro-1-({(cyanoimino)[(2,6-dimethoxy-3-pyridinyl)amino]methyl}amino)propyl]benzamide;
4-chloro-N-[2,2-dichloro-1-({(cyanoimino)[(2-methoxy-3-pyridinyl)amino]methyl}amino)propyl]benzamide; and
4-chloro-N-(2,2-dichloro-1-{[[(2-chloro-3-pyridinyl)amino](cyanoimino)methyl]amino}propyl)benzamide.

14. The method according to claim 1, wherein
R₁ is heterocycle;
R₂ is haloalkyl; and
R₃ is arylalkyl.

15. The method according to claim 1 wherein
R₁ is heterocycle wherein the heterocycle is pyridinyl optionally substituted with 1 or 2 substituents independently selected from the group consisting of alkoxy and halogen;
R₂ is haloalkyl wherein the haloalkyl is 1,1-dichloroethyl; and
R₃ is arylalkyl wherein the aryl of the arylalkyl is selected from the group consisting of naphthyl and phenyl, wherein the phenyl is optionally substituted with 1 or 2 substituents indpendently selected from the group consisting of alkoxy, alkyl, alkylthio, cyano, ethylenedioxy, halogen, methylenedioxy, and R_{A}R_{B}N—.

13. The method according to claim 12 wherein the compound is N-[2,2-dichloro-1-({(cyanoimino)[(2-methoxy-3-pyridinyl)amino]methyl}amino)propyl]-2-(3,4-dimethoxyphenyl)acetamide.

14. A pharmaceutical composition comprising a therapeutically effective amount of a compound of formula I according to claim 1 in combination with a pharmaceutically acceptable carrier.
